(19) United States
(12) Patent Application Publication
(10) Pub. No.: US 2026/0216046 A1
(43) Pub. Date: Jul. 30, 2026

(54) SURFACTANT FREE PERSONAL CARE COMPOSITIONS

(71) Applicant: THE PROCTER & GAMBLE COMPANY, Cincinnati, OH (US)

(72) Inventors: Robin Lynn MCKIERNAN, Mason, OH (US); Yonas GIZAW, West Chester, OH (US); Gary Allen ECHLER, Cincinnati, OH (US); Genevieve Cagalawan WENNING, Villa Hills, KY (US); Steven Daryl SMITH, Fairfield, OH (US)

(21) Appl. No.: 19/629,597

(22) Filed: Mar. 26, 2026

Related U.S. Application Data
(63) Continuation of application No. 17/516,743, filed on Nov. 2, 2021.
(60) Provisional application No. 63/109,404, filed on Nov. 4, 2020.

(51) Int. Cl. A61K 8/81; A61K 8/73; A61Q 5/02

(52) U.S. Cl. CPC A61K 8/8158; A61K 8/737; A61Q 5/02; A61K 2800/5426

(57) ABSTRACT

Personal care compositions containing little or no surfactant and a cationic polymer.

### CROSS REFERENCE TO RELATED APPLICATION

This application is a continuation of U.S. patent application Ser. No. 17/516,743 filed on Nov. 2, 2021, which claims the benefit, under 35 U.S.C. § 119 (e), of U.S. Provisional Patent Application Ser. No. 63/109,404 filed on Nov. 4, 2020, the entire disclosures of all of which are fully incorporated by reference herein.

### FIELD

The present disclosure relates to personal care compositions containing little or no surfactant and a cationic polymer. More particularly the present disclosure relates to personal care compositions for gentle cleaning and conditioning of body surfaces including hair and skin without the use of surfactants that strip the body of its natural protective oils.

### BACKGROUND

In general, a shampoo composition contains a surfactant as a main ingredient and further contains additives such as a preservative, a fragrance, etc. and water. It is common practice to use shampoo compositions based essentially on standard surfactants, such as anionic, nonionic and/or amphoteric type surfactants, but more particularly of anionic type, to clean and wash hair. Anionic surfactants are useful in shampoo compositions; however, they can be problematic in that they can facilitate hair damage or cause irritation and promote color fading from dyed hair due to excessive cleansing ability. In addition, a nonionic surfactant is often used for solubilization and emulsification (or dispersion).

These compositions are applied to wet hair and the lather generated by massaging or rubbing with the hands removes, after rinsing with water, the various types of soiling which are initially present on the hair. Admittedly, these base compositions have good washing power, but the intrinsic cosmetic properties associated with them nevertheless remain fairly poor, owing in particular to the fact that the relatively aggressive nature of such a cleaning treatment can, in the long run, lead to more or less pronounced damage to the hair fiber, this damage being associated in particular with the gradual removal of the lipids or proteins contained in or on the surface of this fiber.

Thus, in order to improve the cosmetic properties of the above detergent compositions, and more particularly those which are to be applied to sensitized hair (i.e. hair which has been damaged or made brittle, in particular under the chemical action of atmospheric agents and/or hair treatments such as permanent-waving, dyeing or bleaching), it is now common practice to introduce to the cleaning routine additional cosmetic agents in the form of conditioning formulations (conditioners), these conditioners being intended mainly to repair or limit the harmful or undesirable effects induced by the various treatments or aggressions to which the hair fibers are subjected more or less repeatedly. These conditioners may, of course, also improve the cosmetic behavior of natural hair.

Thus, to provide sufficient hair cleaning, but without excessive damage to the hair during the cleaning routine a second treatment with a conditioner is needed, which requires additional expense and time. Therefore, what is needed is a single composition that provides sufficient cleaning without damaging the hair.

### SUMMARY

A personal care composition is provided that comprises about 1% to about 10% cationic polymer, wherein the cationic polymer has a molecular weight of greater than about 400,000, a charge density of about 0.4 meq/g to about 4 meq/g, and a surface tension of greater than about 45 mN/m; wherein the composition has a viscosity of about 500 cps to about 30,000 cps; wherein the composition is substantially surfactant free; wherein the composition removes at least about 45% or more artificial sebum as measured by the SYRINGE FILTER POLYMER CLEANING PROCEDURE.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a picture showing a population of hair switches.

FIG. 2 is a picture of a tenpet pad.

FIG. 3 is a diagram of a syringe pump.

FIG. 4 is a diagram of a syringe with a filter.

FIG. 5 is a picture of a filter.

### DETAILED DESCRIPTION

The present invention provides a personal care composition, for both humans and animals, having substantially no surfactant, which includes shampoos, body washes, hair treatments, toothpaste and shaving compositions, where the cleaning benefit is achieved through the addition of cationic polymers using a process of controlled emulsification. The cationic polymer displaces sebum/oil on charged surfaces such as hair, skin and teeth. For example, hair is a complex keratin fiber, which basically consists of three layers: the medulla, the cortex, and the cuticle. To understand the effect of hair care products, the surface charge of the hair has to be taken into closer consideration. Untreated human hair has a strongly negative surface charge. Carboxyl groups of glutamine and aspartic acid and sulfonic acid groups in the hair are responsible for this property. The personal care compositions also have the additional benefits of providing surface (skin, hair and teeth) nourishment, surface (skin, hair and teeth) feel benefits, and hair styling benefits, as well as being gentler in skin mildness assays.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

All numerical parameters are to be understood as being prefaced and modified in all instances by the term “about” unless otherwise indicated. At the very least, and not as an attempt to limit the application of the doctrine of equivalents to the scope of the claims, each numerical parameter described herein should be construed in light of the number of reported significant digits and by applying ordinary rounding techniques.

Also, any numerical range recited herein is intended to include all sub-ranges of the same numerical precision subsumed within the recited range. For example, a range of “1.0 to 10.0” is intended to include all subranges including and between the recited minimum value of 1.0 and the recited maximum value of 10.0—that is, having a minimum value equal to or greater than 1.0 and a maximum value equal to or less than 10.0—such as, for example, 1.4 to 7.6 or 8.1 to 9.7. Any maximum numerical limitation in any numerical range recited in this specification is intended to include all lower numerical limitations subsumed therein; and any minimum numerical limitation in any numerical range recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that an amendment expressly reciting any such sub-range would comply with the requirements of 35 U.S.C. § 112 (a).

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the personal care composition.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto a body surface, such as hair, skin and teeth.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Soluble” means at least about 0.1 g of solute dissolves in 100 ml of solvent, at 25° C. and 1 atm of pressure.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

As used herein, the term “fluid” includes liquids and gels.

As used herein, the term “Room Temperature” or “RT”, refers to an average ambient temperature of between about 20° C. to about 25° C.

As used herein, the articles including “a” and “an” when used in a claim, are understood to mean one or more of what is claimed or described.

As used herein, the word “or” when used as a connector of two or more elements is meant to include the elements individually and in combination; for example X or Y, means X or Y or both.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks Vaporub; hair conditioners, hair dyeing and bleaching compositions, mousses, masks, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral or hair cleaning compositions for animals, such as dogs and cats.

The term “teeth”, as used herein, refers to natural teeth as well as artificial teeth or dental prosthesis.

Personal care compositions may exist in different forms. For example, a personal care composition may be in a liquid form. Personal care compositions may also be in a solid form, like in a bar soap or a semi-solid form, like a paste or gel. Solid personal care compositions can be provided in different shapes and forms, like a rectangle, oval or square, and may be in a powder or pellet form, for example. Additionally, solid and semi-solid forms may be combined with a substrate to form an article as described in more detail in U.S. Patent Application Publication Numbers 2012/0246851; 2013/0043145; 2013/0043146; and 2013/0043147.

In certain embodiments the personal care compositions may comprise about 1% or more of cationic polymer (or mixtures of cationic polymers), for example from about 1% to about 5% cationic polymer. In a shampoo context the cationic polymer is used for removing sebum during washing. The cationic polymer can have a medium charge density (CD) of about 0.4 meq/g to about 4 meq/g and high molecular weight of at least about 400,000. The cationic polymer can be either a synthetic copolymer or modified naturally derived polymers; and differs from traditional surfactants due to its surface tension being greater than or equal to 45 mN/m.

While not being limited to theory it is believed the viscosity of the polymer in water (i.e. the final formulation) helps with perfume stability (by increasing the amount of time for the perfume droplets to diffuse together). The charge of the polymer is the opposite-too high a charge can cause coagulation and flocculation with ingredients in the perfume causing the polymer to precipitate out of solution.

It is further believed charge density and molecular weight together provide both the conditioning/hydrating feel/wet slick of the formula and provide the desired sebum cleaning. Viscosity provides the desired feel for the consumers in hand to be able to get the product out of the container and spread it in their hair or on their skin. It also helps with perfume stability. So too low a charge density—doesn't clean. Too high a charge density—doesn't clean. Too low a molecular weight—low viscosity and poor wet feel, and poor cleaning. Too low a viscosity—the composition feels like water and slips through the hands. Too high a viscosity—hard to get formula out of bottle and spread through hair. The compositions of the present invention have a viscosity of from about 500 cps to about 30,000 cps, from about 1,000 cps to about 25,000 cps, from about 3,000 cps to about 20,000 cps, from about 5,000 cps to about 10,000 cps, or from about 7,000 cps to about 10,000 cps; as determined by the VISCOSITY TEST described herein.

Viscosity helps slow down the coalescing of perfume droplets. Cellulose and naturally derived polymers are both means to increase the viscosity and thus improve perfume dispersion stability over time. Perfume microcapsules and soft matter are ways to encapsulate the perfume, which can provide two benefits: 1) in cases of high levels of CC10/other high cationic charged polymers, encapsulation can prevent the negative interaction with charged components in the perfume formulation which can cause instability in the formulation itself and for materials to precipitate out of solution; 2) if the density of the capsule is controlled to match that of the formulation then the perfume should remain as droplets in the formulations and not rise to the top. Eutectics can act as a cosolvent and dissolve the perfume; in addition organic acids such as citric acid or salicyclic acid can help control the ionic strength so that the polymer can adsorb and compatibilize the perfume.

### Test Methods

To determine the properties of a cationic polymer, such as charge density, molecular weight and viscosity the below described tests are used.

### Polymer Charge Density Test

Charge density for polymer samples is determined using the Mutek PCD-05 Travel Version Titrator (BTG, Herrsching, Germany) or equivalent; the Mutek PCD-05 detects the streaming potential of the sample and then the sample is neutralized by titration, as described in detail below.

### Instrument:

Mutek PCD-05 Travel Version Titrator
   touchpanel Ser. No. 90/400,050
   Travel titrator module Ser. No. 90/500,042

### Titration Reagents:

Anionic Titrant: Potassium polyvinyl sulphate PVSK 0.001 eq/L Cat #X20403: lot #M047020-005
Cationic Titrant: Poly-Dadmac 0.001 eq/L Cat #X20403 lot #M047918-006

### QA Sample:

0.1% Lubrizol Merquat 100 PolyDADMAC Solution:
Activity 43%; Lot #4E2422AO; MW=150,000
(0.162 g) of (43%) QS to 70.6 g with distilled water pH=5.61

The QA sample is run each day as a quality control/precision check on the instrument as well as assessing the titration cell cleaning procedure to remove absorbed polymer.

### Cleaning Reagent:

Dissolve 500 g of NaBr into 1.25 L bottle water. Once completely dissolved add 0.5 L Acetone.

### Polymers Dilution:

All polymers tested are diluted down to 0.2% w/w in bottled water. pH measurements are performed on diluted polymers at the time Charge Density Measurements are made. Procedure:
1. Clean Titration Cell with Cleaning Reagent per manufacturer's instructions.

Rinse with copious amounts of tap water followed by 3 rinses with bottle water.
2. Run & Record the Charge Density of the QA sample prior to measuring test polymer solutions.
3. Tare Titration Cell on balance then add test sample directly into Titration Cell recording weight of sample added. QS with bottle water between 10-11 g.
4. Follow manufacturer's procedure for measuring charge density of polymers.
5. Record final milliliter Volume of Titrant used in titration.
6. Clean Titration Cell after each Charge Density measurement.

### Charge Density Calculations:

The total milliliter volume of titrant used is recorded and charge density is calculated by the equation:

$$Meq / g = \frac{vol of titrant ( L ) \times Concentration of titrant ( eq / L ) \times 1000 meq / eq}{Weight of polymer in sample ( g )}$$

### Polymer Molecular Weight Test

Polymer molecular mass can be determined by GPC SEC/MALS when not already supplied by the manufacturer. The High Performance Liquid Chromatography (HPLC) is a Waters Alliance 2695 HPLC (Waters Corporation, Milford MA) auto injector containing a bank of Tosoh columns (TSK gel columns for cationic polymers; Tosoh Bioscience LC, King of Prussia PA) at room temperature. The flow rate is 0.5 mL/min and the mobile phase is 0.1% sodium nitrate in water.

The detectors are Wyatt Dawn EOS Light scattering detector (Wyatt Technology Corporation, Santa Barbara CA) calibrated with toluene and normalized using Bovine Serum Albumin in mobile phase and a Wyatt Optilab rEX refractive index detector at 40° C.

Samples for analysis are prepared at a known concentration in the range of 3 to 5 mg/mL. Samples are filtered using 0.45 μm polypropylene membrane filters. The injection volume is 100 μL. The data is collected and analyzed using ASTRA 5.3.4.14. Values for dn/dc are calculated from the RI trace assuming 100% mass recovery. Weight average molecular weight is reported.

### Viscosity Test

As described below, viscosity is measured at room temperature with the Brookfield DV2TRVTJ0 viscometer (AMETEK Brookfield, Middleboro, MA) or equivalent over five minutes using the Small Sample Adapter and a 27 spindle. The small sample adapter consists of a cylindrical sample chamber and spindle and provides a defined geometry system for accurate viscosity measurements at precise shear rates. The small sample adapter is designed to measure small sample volumes of 2 to 16 ml. The DV2T has the capability of measuring viscosity over an extremely wide range. For example, the DV2TRV can measure fluids within the range of 100-40,000,000 cP.

### Sample Preparation

If there is significant aeration of the product then the air bubbles can be removed by either sonication or centrifugation.

### Preparation of Equipment

Before operation: turn on both the water bath and cooler, making sure that the flow control of the bath is all the way open.
Follow the manufacturers' instructions for start-up, zeroing and gap setting of the viscometer/rheometer.
A calibration check should be run once each day of use or after changing cones.

### Instrument Operation

1. Confirm that the instrument is level (bubble is centered in circle of level indicator on viscometer).
2. Check that the temperature and the cone fitted are correct, (see conditions in the finished product specifications).
3. Select the speed to the appropriate RPM stated in the technical standards (use 1 RPM if not stated).
4. Set to display cps reading.
5. Draw the sample into a disposable plastic syringe and discharge into sample container several times to remove air trapped in syringe.
6. Place the required amount of sample in the middle of the viscometer cup using the disposable plastic syringe (make sure no bubbles are present).
7. Replace the cup, being careful to raise the cup straight up onto the cone and secure the cup without significant movement.
8. Let sample sit for 1 minute before taking the reading to ensure that the temperature of the cone and sample are equilibrated. Some units have a temperature readout for what is in the cup, for those units wait until the reading says required temperature specified in technical standards (which may be shorter or longer). The read-out need not be 0.0 when starting.
9. Set timer for 3 minutes and start viscometer motor.
10. Take viscosity measurement after 3 minutes.
11. Turn off motor and carefully remove cup.

### Surface Tension Test

### Procedure for Measuring Surface Tension Using Kruss 100 Tensiometer

Surface tension is measured using a Kruss Model 100 Tensiometer (Krüss GMBH, Germany) or equivalent and Advance software. A Wilhelmy platinum probe PL01 was used with a wetting length of 40.2 mm. Both surface tension (mN/m) and temperature (° C.) are recorded.

Polymeric samples are tested at a 0.5% aqueous solution. Samples are equilibrated to room temperature (21° C.-24° C.) and then tested in duplicate. Water controls (+1 mN/m of expected value) are run before and after each polymer solution to ensure the platinum probe is thoroughly clean.

$$Expected Value ( water ) = 72.86 mN / m = ( 20 °C . - Temp . ) ( - 0.1514 mN / m / °C . )$$

The compositions of the present invention may include little or no surfactant; and surface tension, which can be used to describe surfactancy should be greater than 45 mN/m. A surfactant is a compound that lowers the surface tension between two liquids, between a gas and a liquid, or between a liquid and a solid. Surfactants adsorb to the air/water interface and reduce the surface tension of water. A surfactant can be defined as a chemical meeting all five of the following criteria: 1) used in detergent, has surface-active properties, and consists of hydrophilic and hydrophobic groups, 2) capable of reducing the surface tension of water to below 45 mN/m, 3) of forming emulsions and/or microemulsions and/or micelles, 4) adsorption at water/solid interface, and 5) forming spreading or adsorption monolayers at the water-air interface. In addition, surfactants generally tend to be lower molecular weight, have hydrophilic and hydrophobic components, and tend to self-assemble into micelles in an aqueous solution, for example Crodacel® and Lamequat®.

### Interfacial Tension Measurement

### Hardware Setup and Calibration

1. Use a video goniometer instrument with backlight, automated syringe injector, and cameracapable of at least 50 frames/see; for example, an EIN11-In Vitro Contact Angle.
2. Fill 1 ml syringe with purified water. Remove all air bubbles from the syringe by pumping the syringe empty in the bottle and then inverting and pushing a drop of solution out.
3. Use appropriate software for analysis, for example the Software for an EIN11, which will be referenced for the below described software steps.
4. Turn on light using knob on silver box next to monitor by twisting in a clockwise direction
5. Attach needle to syringe. Make sure to not touch the tip of the needle.
6. Place needle in round syringe-holder and place in Automated Syringe Injector. Clamp in place with white plastic clamp. Note: It may be necessary to raise the injector bar by sliding the speed control as high as it will go and clicking “Pump in”. Watch the bar rise until it is just above the syringe plunger. Then click “Pump out” to bring the bar down until it touches the plunger.
7. Click check box next to “Video” to turn on camera.
8. Adjust camera position and focus by using three knobs on camera stand. The needle should be vertical and just visible in the top middle of the image.
9. Adjust the lamp up or down as necessary to produce a uniform white background.
10. Pump out a single pendant drop. Observe the drop to ensure it is uniformly dark with a bright spot in the middle. The drop should fill the image as much as possible, without going beyond the bottom of the frame. Picture 2-4
11. It may be necessary to make adjustments to achieve a good picture. If there are reflections on the drop, you can:
Adjust the light intensity
   Adjust the aperture on the lens
Put a large piece of foil around the apparatus to block the light.
   Turn off the overhead light.
12. Click Snapshot to take an image of the water drop.
13. Click Distance box, then click on both sides of the needle to draw a line across. Then click the Calibration tab and enter the Distance in the Measured Distance (mm) box. Enter the actual width in the Actual Distance (mm) box and click Apply.
14. Set Density of light phase to 0.0011. Set Density of water according to the room temperature.
15. In Images tab, click IF Tension button. The Interfacial Tension should be between 70.5-72.8.
16. Under File menu, Click “Save As . . . ” and save file as Water Calibration. RunningIFT
1 Fill a 1 ml syringe with desired aqueous solution. Attach green 0.81 mm needle and repeat steps 4-14 above to load syringe in apparatus.
2. Fill cuvette ¾ full with oil phase.
3. Insert needle into cuvette and adjust stage so needle is centered in cuvette.
4. Refocus camera and recenter needle since the optics will change by adding the cuvette.
5. Place cross on screen about 1 inch below the needle by clicking on the screen.
6. Under Capture tab, check “Video Trigger by Z<120” and “Full Size”. Set the following parameters:

| | |
|---|---|
| Images Before Trigger | 20 |
| Image period before trigger (s) | 0.02 |
| Images after trigger | 550 |
| Initial period after trigger (s) | 0.02 |
| Post-trigger period multiplier | 1.00 |
| Camera Frame Rate: | 50 |

7. Under Pump tab, Check “Start on Run”. Set the following parameters:

| | |
|---|---|
| Manual Rate | 12.567 |
| Automatic Rate | 12.567 |
| Automatic Volume | 7.0 |
| Displacement | 7.0 |
| Total Syringe Capacity | 1000 |
| Syringe Internal Diameter (mm) | 4.55 |
| Syringe Scale Length (mm) | 57.3 |

8. Under Movie tab, click run. If bubble grows larger than image frame, or if the bubble drops off, reduce the Automatic Volume by 1 ul. If drop is too small, increase Automatic Volume by 1 μl. Run will complete and software will automatically switch to analysis mode.

### Movie Analysis

1. In Calibration tab, Change “Density of heavy phase (g/cc)” and “Density of light phase (g/cc) to appropriate values.
2. In Images tab, click “IF Tension”, ensure Tip Width is correct. If not, repeat step 15 from Setup and Calibration.
3. Click “Cineloop”. Make sure “End at Image Number” is 570. Click OK. Software will calculate IFT for all images. This will take several minutes.
4 Once calculations are done, click Graph tab and click “Options”. Set Y2 Axis to Pendant Volume.
5 If everything ran correctly, the volume should rise quickly to a maximum and then plateau.

### Personal Care Compositions

The personal care compositions (or compositions) of the present invention comprise a cationic polymer and one or more of the components listed below.

The personal care compositions may be in the form of solutions, dispersion, emulsions, powders, talcs, encapsulated, spheres, spongers, solid dosage forms, foams, and other delivery mechanisms; and may fall into many consumer product categories, as described above.

### Cationic Polymers

The personal care composition comprises a cationic polymer. These cationic polymers may be naturally derived or naturally derived and then modified. Examples include polysaccharides such as cationic guar, cationic chitosan, cationic dextran, cationic cellulose, cationic cyclodextrin, cationic starch, cationic pectin, cationic polyglucan, and their derivatives. They also include cationic peptides and proteins.

These cationic polymers can include at least one of (a) a cationic guar polymer, (b) a cationic non-guar galactomannan polymer, (c) a cationic tapioca polymer, (d) a synthetic, non-crosslinked, cationic polymer, (e) a cationic cellulose polymer. Additionally, the cationic polymer can be a mixture of cationic polymers.

A synthetic cationic polymer may include several monomeric units, so they may be referred to as a copolymer rather than a homopolymer, which consists of a single type of monomeric unit. An example of a cationic homopolymer includes polyethylenimine. The polymers of the present disclosure may be a random copolymer. In one example, a polymer of the present disclosure may be water-soluble and/or water-dispersible, which means that the polymer does not, over at least a certain pH and concentration range, form a two-phase composition in water at 23° C.±2.2° C. In some embodiments, a polymer of the present invention comprises monomeric units such as those listed below:

a. Nonionic Monomeric Units

The nonionic monomeric units may be selected from the group consisting of: nonionic hydrophilic monomeric units, nonionic hydrophobic monomeric units, and mixtures thereof.

Non-limiting examples of nonionic hydrophilic monomeric units suitable for the present invention include nonionic hydrophilic monomeric units derived from nonionic hydrophilic monomers selected from the group consisting of: hydroxyalkyl esters of α,β-ethylenically unsaturated acids, such as hydroxyethyl or hydroxypropyl acrylates and methacrylates, glyceryl monomethacrylate, α,β-ethylenically unsaturated amides such as acrylamide, N,N-dimethylacrylamide, N,N-dimethylmethacrylamide, N-methylolacrylamide, α,β-ethylenically unsaturated monomers bearing a water-soluble polyoxyalkylene segment of the poly(ethylene oxide) type, such as poly(ethylene oxide) α-methacrylates (Bisomer S20W, S10W, etc., from Laporte) or α,ω-dimethacrylates, Sipomer BEM from Rhodia (@-behenyl polyoxyethylene methacrylate), Sipomer SEM-25 from Rhodia (@-tristyrylphenyl polyoxyethylene methacrylate), α,β-ethylenically unsaturated monomers which are precursors of hydrophilic units or segments, such as vinyl acetate, which, once polymerized, can be hydrolyzed in order to give rise to vinyl alcohol units or polyvinyl alcohol segments, vinylpyrrolidones, α,β-ethylenically unsaturated monomers of the ureido type, and in particular 2-imidazolidinone-ethyl methacrylamide (Sipomer WAM II from Rhodia), and mixtures thereof. In one example, the nonionic hydrophilic monomeric unit is derived from acrylamide.

Non-limiting examples of nonionic hydrophobic monomeric units suitable for the present invention include nonionic hydrophobic monomeric units derived from nonionic hydrophobic monomers selected from the group consisting of: vinylaromatic monomers such as styrene, alpha-methylstyrene, vinyltoluene, vinyl halides or vinylidene halides, such as vinyl chloride, vinylidene chloride, C₁-C₁₂ alkylesters of α,β-monoethylenically unsaturated acids such as methyl, ethyl or butyl acrylates and methacrylates, 2-ethylhexyl acrylate, vinyl esters or allyl esters of saturated carboxylic acids, such as vinyl or allyl acetates, propionates, versatates, stearates, α,β-monoethylenically unsaturated nitriles containing from 3 to 12 carbon atoms, such as acrylonitrile, methacrylonitrile, α-olefins such as ethylene, conjugated dienes, such as butadiene, isoprene, chloroprene, and mixtures thereof.

b. Cationic Monomeric Units

Non-limiting examples of cationic monomeric units suitable for the present invention include amine containing monomeric units derived from monomers selected from the group consisting of: N,N-(dialkylamino-ω-alkyl)amides of α,β-monoethylenically unsaturated carboxylic acids, such as N,N-dimethylaminomethyl-acrylamide or -methacrylamide, 2-(N,N-dimethylamino)ethylacrylamide or -methacrylamide, 3-(N,N-dimethylamino) propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino) propyl methacrylate, 2-(tert-butylamino)ethyl methacrylate, 2-(dipentylamino)ethyl methacrylate, and 2 (diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium (3-propylmethacrylamide methyl sulfate, methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride (METAC) or methyl sulfate, and acryloyloxyethyltrimethylammonium chloride (AETAC); 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N1-(3-(2 ((3-methacrylamidopropyl)dimethylammino)-acetamido)propyl)-N1, N1, N3, N3, N3 pentamethylpropane-1,3-diaminium chloride (TRIQUAT or TQ), and mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In embodiments, the non-ionic monomers are selected from acrylamide derivatives from the group consisting of: acrylamide, mono-alkyl substituted acrylamide, symmetrical or asymmetrical, di-N-alkyl substituted acrylamide derivatives, methacrylamide, mono-alkyl substituted methacrylamide, symmetrical or asymmetrical, di-N-alkyl substituted methacrylamide derivatives and mixtures thereof.

In another example, the acrylamide derivatives of the present invention are selected from the group consisting of: N,N-dimethylacrylamide (NDMAAM), acrylamide, methyl acrylamide, ethylacrylamide, N,N-diethylacrylamide, methacrylamide, N,N-dimethyl methacrylamide, and mixtures thereof.

Further examples of cationic monomeric units suitable for the present invention include cationic monomeric units derived from cationic monomers selected from the group consisting of: N,N-(dialkylamino-ω-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino) propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino) propyl methacrylate, 2-(tert-butylamino)ethyl methacrylate, 2-(dipentylamino)ethyl methacrylate, and 2 (diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N1-(3-(2((3-methacrylamidopropyl)dimethylammino)-acetamido)propyl)-N¹, N¹, N³, N³, N³-pentamethylpropane-1,3-diaminium chloride (TRIQUAT or TQ), and mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In embodiments, the cationic monomeric units are derived from cationic monomers selected from the group consisting of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

In embodiments, the cationic monomeric units are derived from cationic monomers selected from the group consisting of: trimethylammonium ethyl (meth)acrylate bromide, chloride or methyl sulfate, trimethylammonium ethyl (meth)acrylate bromide, chloride or methyl sulfate, trimethylammonium ethyl (meth)acrylate bromide, chloride or methyl sulfate, dimethylaminoethyl (meth)acrylate benzyl chloride, 4-benzoylbenzyl dimethylammoniumethyl (meth)acrylate bromide, chloride or methyl sulfate, trimethylammonium ethyl (meth)acrylamido bromide, chloride, or methyl sulfate, trimethylammonium propyl (meth)acrylamido braomide, chloride, or methyl sulfate, vinyl benzyl trimethyl ammonium bromide, chloride or methyl sulfate, diallyldimethyl ammonium chloride, 1-ethyl-2-vinylpyridinium bromide, chloride or methyl sulfate, 4-vinylpyridinium bromide, chloride or methyl sulfate, and mixtures thereof.

The personal care composition may comprise a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β (1-4) glycosidic linkages. The galactose branching arises by way of an a (1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

The cationic polymer, may include but is not limited to a cationic guar polymer; wherein a guar polymer may have a weight average molecular weight of less than about 10 million g/mol, or from about 400 thousand to about 10 million g/mol, or from about 500 thousand to about 5 million g/mol, or from about 750 thousand to about 3 million g/mol, or from about 1 million to about 2 million g/mol. The cationic guar polymer may have a charge density of from about 0.4 to about 4.0 meq/g, or from about 0.6 to about 3.0 meq/g, or from about 0.75 to about 2.5 meq/g; or from about 1.0 meq/g to about 2.0 meq/g.

Suitable cationic guar polymers include cationic guar gum derivatives, such as guar hydroxypropyltrimonium chloride. The cationic guar polymer may be a guar hydroxypropyltrimonium chloride. Specific examples of guar hydroxypropyltrimonium chlorides include the Jaguar® series commercially available from Solvay, for example Jaguar® C-500, commercially available from Solvay. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.3 meq/g and a molecular weight of about 500,000 g/mol and is available from Solvay as Jaguar® Optima. Other suitable guar hydroxypropyltrimonium chloride are: guar hydroxypropyltrimonium chloride which has a charge density of about 0.7 meq/g and a molecular weight of about 1,500,000 g/mol and is available from Solvay as Jaguar® Excel. Other suitable guar hydroxypropyltrimonium chloride are: guar hydroxypropyltrimonium chloride which has a charge density of about 1.1 meq/g and a molecular weight of about 500,000 g/mol and is available from ASI.

Other suitable guar hydroxypropyltrimonium chloride are: Hi-Care 1000, which has a charge density of about 0.7 meq/g and a molecular weight of about 600,000 g/mole and is available from Solvay; N-Hance 3269 and N-Hance 3270, which have a charge density of about 0.7 meq/g and a molecular weight of about 425,000 g/mol and are available from ASI; N-Hance 3196, which has a charge density of about 0.8 meq/g and a molecular weight of about 1,100,000 g/mol and is available from ASI. BF-13, which is a borate (boron) free guar of charge density of about 1.1 meq/g and molecular weight of about 800,000 and BF-17, which is a borate (boron) free guar of charge density of about 1.7 5 meq/g and molecular weight of about 800,000 both available from ASI. Another suitable guar hydroxypropyltrimonium chloride is Dehyquart Guar HP available from BASF.

The personal care compositions of the present invention may comprise a galactomannan polymer derivative having a mannose to galactose ratio of greater than 2:1 on a monomer to monomer basis, the galactomannan polymer derivative selected from the group consisting of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an a (1-6) linkage. The ratio of mannose monomers to galactose monomers varies according to the species of the plant and also is affected by climate. Non-Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a molecular weight from about 400,000 g/mol to about 10,000,000 g/mol, and/or from about 500,000 g/mol to about 5,000,000 g/mol.

The personal care compositions of the invention can also include galactomannan polymer derivatives which have a cationic charge density from about 0.4 meq/g to about 4.0 meq/g. The galactomannan polymer derivatives may have a cationic charge density from about 0.6 meq/g to about 4 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds.

Alternatively, the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose greater than about 4:1, a molecular weight of about 400,000 g/mol to about 10,000,000 g/mol, and/or from about 500,000 g/mol to about 10,000,000 g/mol, and/or from about 750,000 g/mol to about 3,000,000 g/mol, and/or from about 1,000,000 g/mol to about 2,000,000 g/mol and a cationic charge density from about 0.4 meq/g to about 4 meq/g, and/or from 0.6 meq/g to about 3 meq/g and can be derived from a Cassia plant.

The personal care compositions can comprise water-soluble cationically modified starch polymers. As used herein, the term “cationically modified starch” refers to a starch to which a cationic group is added prior to degradation of the starch to a smaller molecular weight, or wherein a cationic group is added after modification of the starch to achieve a desired molecular weight. The definition of the term “cationically modified starch” also includes amphoterically modified starch. The term “amphoterically modified starch” refers to a starch hydrolysate to which a cationic group and an anionic group are added.

The cationically modified starch polymers for use in the personal care compositions can have a molecular weight of greater than or equal to 400,000 molecular weight.

The personal care compositions can include cationically modified starch polymers which have a charge density of from about 0.4 meq/g to about 4.0 meq/g, and/or from about 0.6 meq/g to about 3 meq/g. The chemical modification to obtain such a charge density includes, but is not limited to, the addition of amino and/or ammonium groups into the starch molecules. Non-limiting examples of these ammonium groups may include substituents such as hydroxypropyl trimmonium chloride, trimethylhydroxypropyl ammonium chloride, dimethylstearylhydroxypropyl ammonium chloride, and dimethyldodecylhydroxypropyl ammonium chloride. See Solarek, D. B., Cationic Starches in Modified Starches: Properties and Uses, Wurzburg, O. B., Ed., CRC Press, Inc., Boca Raton, Fla. 1986, pp 113-125. The cationic groups may be added to the starch prior to degradation to a smaller molecular weight or the cationic groups may be added after such modification.

The source of starch before chemical modification can be chosen from a variety of sources such as tubers, legumes, cereal, and grains. Non-limiting examples of this source starch may include corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures thereof.

The cationically modified starch polymers can be selected from degraded cationic maize starch, cationic tapioca, cationic potato starch, and mixtures thereof. Alternatively, the cationically modified starch polymers are cationic corn starch and cationic tapioca.

The starch, prior to degradation or after modification to a smaller molecular weight, may comprise one or more additional modifications. For example, these modifications may include cross-linking, stabilization reactions, phosphorylations, and hydrolyzations. Stabilization reactions may include alkylation and esterification.

The cationically modified starch polymers may be incorporated into the composition in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via the thermo-mechanical energy input of the processing equipment), or combinations thereof.

Suitable cationically modified starch for use in personal care compositions are available from known starch suppliers. Also suitable for use in personal care compositions are nonionic modified starch that can be further derivatized to a cationically modified starch as is known in the art. Other suitable modified starch starting materials may be quaternized, as is known in the art, to produce the cationically modified starch polymer suitable for use in personal care compositions.

The synthetic cationic polymers of the present invention can be made by a wide variety of techniques, including bulk, solution, emulsion, or suspension polymerization. Polymerization methods and techniques for polymerization are described generally in Encyclopedia of Polymer Science and Technology, Interscience Publishers (New York), Vol. 7, pp. 361-431 (1967), and Kirk-Othmer Encyclopedia of Chemical Technology, 3rd edition, Vol 18, pp. 740-744, John Wiley & Sons (New York), 1982, both incorporated by reference herein. See also Sorenson, W. P. and Campbell, T. W., Preparative Methods of Polymer Chemistry. 2nd edition, Interscience Publishers (New York), 1968, pp. 248-251, incorporated by reference herein, for general reaction techniques suitable for the present invention. In one example, the polymers are made by free radical copolymerization, using water soluble initiators. Suitable free radical initiators include, but are not limited to, thermal initiators, redox couples, and photochemical initiators. Redox and photochemical initiators may be used for polymerization processes initiated at temperatures below about 30° C. (86° F.). Such initiators are described generally in Kirk-Othmer Encyclopedia of Chemical Technology, 3rd edition, John Wiley & Sons (New York), Vol. 13, pp. 355-373 (1981), incorporated by reference herein. Typical water soluble initiators that can provide radicals at 30° C. or below include redox couples, such as potassium persulfate/silver nitrate, and ascorbic acid/hydrogen peroxide. In one example, the method utilizes thermal initiators in polymerization processes conducted above 40° C. (104° F.). Water soluble initiators that can provide radicals at 40° C. (104° F.) or higher can be used. These include, but are not limited to, hydrogen peroxide, ammonium persulfate, and 2,2′-azobis(2-amidinopropane) dihydrochloride. In one example, water soluble starting monomers are polymerized in an aqueous alcohol solvent at 60° C. (140° F.) using 2,2′-azobis(2-amidinopropane) dihydrochloride as the initiator.

### Liquid Personal Care Compositions

Liquid personal care compositions may include an aqueous carrier, which can be present at a level of from about 90% or greater. The aqueous carrier may comprise water, or a miscible mixture of water and organic solvent. Non-aqueous carrier materials may also be employed.

The personal care composition may be applied by a variety of means, including by rubbing, wiping or dabbing with hands or fingers, or by means of an implement and/or delivery enhancement device. Non-limiting examples of implements include a sponge or sponge-tipped applicator, a mesh shower puff, a swab, a brush, a wipe (e.g., wash cloth), a loofah, and combinations thereof. Non-limiting examples of delivery enhancement devices include mechanical, electrical, ultrasonic and/or other energy devices. The personal care composition may be sold together with such an implement or device. Alternatively, an implement or device can be sold separately but contain indicium to indicate usage with a personal care composition. Implements and delivery devices can employ replaceable portions (e.g., the skin interaction portions), which can be sold separately or sold together with the personal care composition in a kit.

### Optional Ingredients

In the present invention, a personal care composition may further comprise one or more optional ingredients, including benefit agents. Suitable benefit agents include, but are not limited to conditioning agents, anti-dandruff agents, chelating agents, and natural oils such as sunflower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, sensates, anti-dandruff agents, propellants, solvents, diluents, anti-oxidants, vitamins and combinations thereof. In the present invention, the composition may have from about 0.5% to about 2% of a perfume.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

### Chelating Agents

Personal care compositions of the present invention can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties.

Chelating agents can be incorporated in the compositions herein in amounts ranging from 0.001% to 10.0% by weight of the total composition, preferably 0.01% to 2.0%.

Nonlimiting chelating agent classes include carboxylic acids, aminocarboxylic acids, including aminocids, phosphoric acids, phosphonic acids, polyphosphonic acids, polyethyleneimines, polyfunctionally-substituted aromatic, their derivatives and salts.

Nonlimiting chelating agents include the following materials and their salts. Ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic acid, aspartic acid, glutamic acid, glycine, malonic acid, histidine, diethylenetriaminepentaacetate (DTPA), N-hydroxyethylethylenediaminetriacetate, nitrilotriacetate, ethylenediaminetetrapropionate, triethylenetetraaminehexaacetate, ethanoldiglycine, propylenediaminetetracetic acid (PDTA), methylglycinediacetic acid (MODA), diethylenetriaminepentaacetic acid, methylglycinediacetic acid (MGDA), N-acyl-N,N′,N′-ethylenediaminetriacetic acid, nitrilotriacetic acid, ethylenediaminediglutaric acid (EDGA), 2-hydroxypropylenediamine disuccinic acid (HPDS), glycinamide-N,N′-disuccinic acid (GADS), 2-hydroxypropylenediamine-N—N′-disuccinic acid (HPDDS), N-2-hydroxyethyl-N,N-diacetic acid, glyceryliminodiacetic acid, iminodiacetic acid-N-2-hydroxypropyl sulfonic acid, aspartic acid N-carboxymethyl-N-2-hydroxypropyl-3-sulfonic acid, alanine-N,N′-diacetic acid, aspartic acid-N,N′-diacetic acid, aspartic acid N-monoacetic acid, iminodisuccinic acid, diamine-N,N′-dipolyacid, monoamide-N,N′-dipolyacid, diaminoalkyldi (sulfosuccinic acids) (DDS), ethylenediamine-N—N′-bis(ortho-hydroxyphenyl acetic acid)), N,N′-bis(2-hydroxybenzyl)ethylenediamine-N, N′-diacetic acid, ethylenediaminetetraproprionate, triethylenetetraaminehexacetate, diethylenetriaminepentaacetate, dipicolinic acid, ethylenedicysteic acid (EDC), ethylenediamine-N,N′-bis(2-hydroxyphenylacetic acid) (EDDHA), glutamic acid diacetic acid (GLDA), hexadentateaminocarboxylate (HBED), polyethyleneimine, 1-hydroxydiphosphonate, aminotri(methylenephosphonic acid) (ATMP), nitrilotrimethylenephosphonate (NTP), ethylenediaminetetramethylenephosphonate, diethylenetriaminepentamethylenephosphonate (DTPMP), ethane-1-hydroxydiphosphonate (HEDP), 2-phosphonobutane-1,2,4-tricarboxylic acid, polvphosphoric acid, sodium tripolyphosphate, tetrasodium diphosphate, hexametaphosphoric acid, sodium metaphosphate, phosphonic acid and derivatives, Aminoalkylen-poly (alkylenphosphonic acid), aminotri (1-ethylphosphonic acid), ethylenediaminetetra(1-ethylphosphonic acid), aminotri (1-propylphosphonic acid), aminotri (isopropylphosphonic acid), ethylenediaminetetra(methylenephosphonic acid) (EDTMP), 1,2-dihydroxy-3,5-disulfobenzene.

The carrier useful the personal care compositions of the present invention may include water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

Personal care compositions can also include one or more humectants. Examples of such humectants can include polyhydric alcohols. Further, humectants such as glycerin can be included the personal care composition as a result of production or as an additional ingredient. Including additional humectant can result in a number of benefits such as improvement in hardness of the personal care composition, decreased water activity of the personal care composition, and reduction of a weight loss rate of the personal care composition over time due to water evaporation.

### Foam Dispenser

A personal care composition of the present invention described herein may be provided in a foam dispenser. The foam dispenser may be an aerosol foam dispenser. The aerosol foam dispenser may comprise a reservoir for holding the personal treatment composition. The reservoir may be made out of any suitable material selected from the group consisting of plastic, metal, alloy, laminate, and combinations thereof. And the reservoir may be for one-time use. The reservoir may be removable from the aerosol foam dispenser. Alternatively, the reservoir may be integrated with the aerosol foam dispenser. And there may be two or more reservoirs.

The foam dispenser may also be a mechanical foam dispenser. The mechanical foam dispenser described may be selected from the group consisting of squeeze foam dispensers, pump foam dispensers, other mechanical foam dispensers, and combinations thereof. The mechanical foam dispenser may be a squeeze foam dispenser. Non-limiting examples of suitable pump dispensers include those described in WO 2004/078903, WO 2004/078901, and WO 2005/078063 and may be supplied by Albea (60 Electric Ave., Thomaston, CT 06787 USA) or Rieke Packaging Systems (500 West Seventh St., Auburn, Indiana 46706).

The mechanical foam dispenser may comprise a reservoir for holding the personal treatment composition. The reservoir may be made out of any suitable material selected from the group consisting of plastic, metal, alloy, laminate, and combinations thereof. The reservoir may be a refillable reservoir such as a pour-in or screw-on reservoir, or the reservoir may be for one-time use. The reservoir may also be removable from the mechanical foam dispenser. Alternatively, the reservoir may be integrated with the mechanical foam dispenser. And there may be two or more reservoirs.

The reservoir may be comprised of a material selected from the group consisting of rigid materials, flexible materials, and combinations thereof. The reservoir may be comprised of a rigid material if it does not collapse under external atmospheric pressure when it is subject to an interior partial vacuum.

### Propellant or Blowing Agent

The personal care composition described herein may comprise from about from about 1% to about 10% propellant or blowing agent, alternatively from about 2% to about 8% propellant, by weight of the personal care composition.

The propellant or blowing agent may comprise one or more volatile materials, which in a gaseous state, may carry the other components of the personal care composition in particulate or droplet form or as a foam. The propellant or blowing agent may have a boiling point within the range of from about −45° C. to about 5° C. The propellant or blowing agent may be liquefied when packaged in convention aerosol containers under pressure. The rapid boiling of the propellant or blowing agent upon leaving the aerosol foam dispenser may aid in the atomization or foaming of the other components of the personal care composition.

Aerosol propellants or blowing agents which may be employed in an aerosol composition of the present invention may include the chemically-inert hydrocarbons such as propane, n-butane, isobutane, cyclopropane, and mixtures thereof, as well as halogenated hydrocarbons such as dichlorodifluoromethane, 1,1-dichloro-1,1,2,2-tetrafluoroethane, 1-chloro-1,1-difluoro-2,2-trifluoroethane, 1-chloro-1,1-difluoroethane, 1,1-difluoroethane, dimethyl ether, monochlorodifluoromethane, trans-1,3,3,3-tetrafluoropropene, and mixtures thereof. The propellant or blowing agent may comprise hydrocarbons such as isobutane, propane, and butane—these materials may be used for their low ozone reactivity and may be used as individual components where their vapor pressures at 21.1° C. range from about 1.17 Bar to about 7.45 Bar, alternatively from about 1.17 Bar to about 4.83 Bar, and alternatively from about 2.14 Bar to about 3.79 Bar.

### Applicator

In the present invention, personal care composition may be dispensed from an applicator for dispensing directly to the scalp area. Dispensing directly onto the scalp via a targeted delivery applicator enables deposition of the non-diluted cleaning agents directly where the cleaning needs are highest. This also minimizes the risk of eye contact with the cleansing solution.

The applicator is attached or can be attached to a bottle containing the cleansing personal care composition. The applicator can consist of a base that holds or extends to a single or plurality of tines. The tines have openings that may be at the tip, the base or at any point between the tip and the base. These openings allow for the product to be distributed from the bottle directly onto the hair and/or scalp.

Alternatively, the applicator can also consist of brush-like bristles attached or extending from a base. In this case product would dispense from the base and the bristles would allow for product distribution via the combing or brushing motion.

Applicator and tine design and materials can also be optimized to enable scalp massage. In this case it would be beneficial for the tine or bristle geometry at the tips to be more rounded similar to the roller ball applicator used for eye creams. It may also be beneficial for materials to be smoother and softer; for example, metal or metal-like filaments.

### EXAMPLES

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

### Example 1

The following procedure was used in EXAMPLE 1 of the present invention.

### Procedure for Washing Sebumed Hair Switches

Sink conditions are water temperature 100±5° F., 47 psi water pressure, and ~1.5 gallons per minute water flow rate. All samples are tested at 5% active unless otherwise noted.

### 1) Prewash:

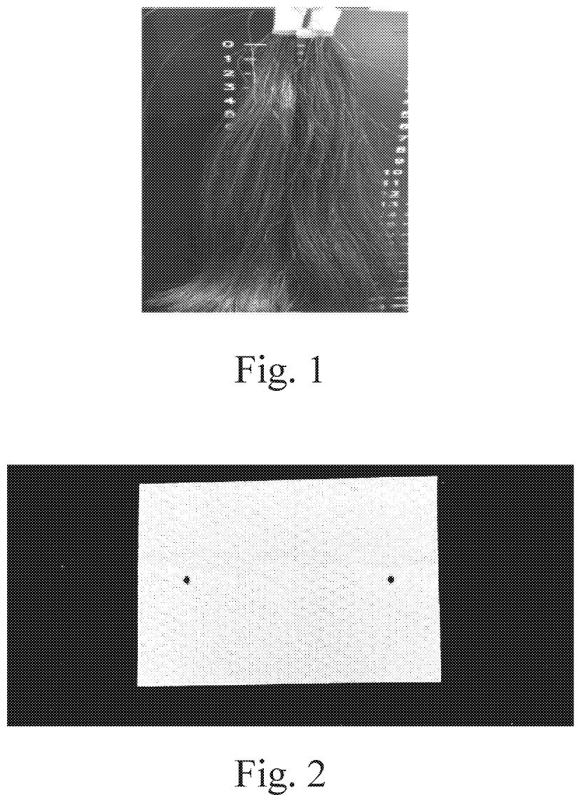

4 gram, 8 inch general population hair switches (net round with epoxy and tape, as shown in FIG. 1) from International Hair Importers, Glendale, NY, (catalog #GP-FN-3R) is prewashed per the Global Standard Wash Method listed below.

They are washed per the Global Standard Wash Method.

### Global Standard Wash Method.

1. Adjust water temperature to 100*5″F; Pressure 47 psi; water flow rate to ~1.5 gpm (gallons per minute).
2. Use 0.1 cc global wash (Pantene Pro-V Sheer volume) per 1-gram hair.
3. Place hair switch(es) into switch clamp.
4. Wet/rinse hair thoroughly with water,
5. Apply appropriate amount of global wash (Pantene Pro-v Sheer volume) to front of switch(es); milk for 30 seconds,
6, Rinse for 30 seconds with water.
7. Turn switch(es) around to have back of switch facing forward.
8. Apply appropriate amount of global wash (Pantene Pro-v Sheer volume) to front of switch(es); milk for 30 seconds, then rinse for 30 seconds.
9. Comb hair switch with large teeth 5 times, then with fine teeth of comb 3 times.
10. Rinse hair switch(es) with water for 2 minutes,
11. Hang hair switches on cart to air dried in a CTR (50% RH/70F) for 24 hours prior to use.

### 2) Sebum Application:

Artificial sebum from Advanced Testing Laboratory, Inc. (Cincinnati, OH) is warmed to 37° C. to liquify it first with the aid of a Pro-Wax 100 water bath.

A Tenpet pad (see FIG. 2) is cut to be 3-inch length by 2-inch height and marked with two dots on the smooth side (1 inch from top and bottom height and ½-inch from each end of width creating 2 points 2 inches apart). Artificial ATL sebum is applied to the nonwoven Tenpet pad (received from PGI in rolls) material by applying from dot to dot in a straight line, and then the pad is folded in half around the hair switch so that the two dots touch and then rubbed down the hair multiple times until the desired amount of sebum (98 mg-105 mg) is applied to the hair. The weight of the sebum Tenpet pad is checked periodically until the desired amount of sebum is applied to the hair. There is an approximately one-hour interval between when sebum is applied and Step 3) below. All sebum treated hair and washing should take place in the same day.

### 3) Hair Wash Procedure:

A. Wet hair switch by holding under running water (47 psi) at a rate of 1.5 gmp (gallon per minute) and a temperature of 100 F for 30 sec, milking hair switch while holding under water.
B. Remove hair switch from water flow. 0.4 gms of cleaning solution is applied via a 1 ml pipet evenly down the length of the 4 g hair switch.
C. Out of the water flow, the cleaning solution is milked (using both thumb and index fingers of both hands) into the hair swatch for 30 sec.
D. Place hair back in water flow and while milking, rinse hair under running water for 30 sec.
   a. Excess water squeezed out of 4 g hair switch twice.
E. Hair switch is dried in oven for 45 min at 60° C. Remove hair from oven and hang at room temp.
F. Dry hair switches are cut at the net round epoxy taped end and inserted into a 40 ml vial.
   4) Sebum Extraction from Hair:

### Stock Solutions

Reference Stock (RFS) is prepared by adding 1±0.2 grams of artificial sebum and then 15±0.2 grams of hexane (~22 mL) into a 40 mL scintillation vial. The vial is capped and swirled to help dissolve the sebum.

Check Stock (CKS) is prepared by adding 1±0.2 grams of artificial sebum and then 15±0.2 grams of hexane (~22 mL) into a 40 mL scintillation vial. The vial is capped and swirled to help dissolve the sebum.

1 mL of RFS or CKS is used to measure density.

Internal Standard Stock (NTS) is prepared by adding 0.11±0.01 of nonadecanoic acid, then 0.11±0.01 grams of squalene, then 15±0.2 grams of hexane into a 40 mL scintillation vial. The vial is capped, mixed and warmed (if needed) to help dissolve the solids.

### Samples

The dry hair switches are cut at the net round epoxy taped end. They are placed in a CTCH room (40% RH, 22° C.) overnight and then weighed.

Treated Samples are prepared by placing each cut ~4 gram dried hair switch (previously washed and cleaned as describe above) into a separate empty 40 mL scintillation vial. The weight of each hair switch is recorded.

Check Blank Samples (CBS) are prepared from a minimum of four hair switches that were never sebumed and placed into four separate empty 40 mL scintillation vials. The weight of each hair switch is recorded.

Check Samples (CKS) are prepared from a minimum of five hair switches that were never sebumed and placed into five separate empty 40 mL scintillation vials. The weight of each hair switch is recorded. 0.15 mL of CKS stock is pipette to the side of the control hair switch vial (not directly onto the hair). These vials are processed along with the treated switch samples.

System Blank (SB) is prepared by setting aside an empty vial of the same size and type as the hair switch samples. This vial is not spiked with internal standard (so step 1 is skipped below) but otherwise goes through the hair extraction process as shown below.

The extraction process must be completed within 8 hours after the first hair sample is extracted with hexane. Do not let the hair sit in hexanes for more than 90 minutes during any extraction leg. Typically the hair should be exposed to hexane for about 20 minutes per extraction leg.

### Extraction:

A. Pipet 100 μL of the Internal Standard Stock (NTS) into each CBS, CKS, and treated hair switch vial except for the Internal Blank (SB) which does not get the NTS.
B. Add 30 mL of hexane to each vial including the Internal Blank (SB).
C. Vortex each vial at medium speed (~1700 rpm) for 5 minutes. Do not pulse nor sonicate.
D. Carefully decant each supernatant into a second empty vial marked with the same ID.
E. Dry each supernatant vial under nitrogen on a warm plate set at 30° C. Continue to dryness.
F. Once dry, add 5 mL hexane and vortex.

Curve Standards (RFS) are prepared by adding the following components to 20 mL scintillation vials:

| RFS Added | Add 5.0 mL | Add 0.1 mL NTS | ID/Label |
|---|---|---|---|
| (μL) | Hexanes (mL) | (Int Stand) (μL) | Solution Made |
| 250 | 5.000 | 0.100 | System Suitability |
| | | | (SST) |
| 0 | 5.000 | 100 | RFS-0 |
| 20 | 5.000 | 100 | RFS-20 |
| 65 | 5.000 | 100 | RFS-65 |
| 120 | 5.000 | 100 | RFS-120 |
| 180 | 5.000 | 100 | RFS-180 |
| 250 | 5.000 | 100 | RFS-250 |
| 500 | 5.000 | 100 | RFS-500 |
| 1000 | 5.000 | 100 | RFS-1000 |
| 1500 | 5.000 | 100 | RFS-1500 |
| 3000 | 5.000 | 100 | RFS-3000 |

System Suitability Test (SST) is treated as a QC check. It is injected out of the same vial each time and is injected five times prior to the STD 0. And the SST is rejected after a maximum of 10 study samples. The SST is reinjected at least once at the end of the batch.

### Analytical Sample Prep:

All samples are treated the same from this point on. Vials used are either the Waters Maximum Recovery vials (Part #6000000749cv) or the Waters Clear LCMS Certified vials (Part #6000000751 cv).
A. Aliquot 0.1 mL of reconstituted hair treatments, the curve solutions (RFS), the system suitability (SST), system blank (SB), the check blank (CBS), and the check samples (CKS) into labeled autosampler vials.
B. Make up at least 2 vials of the System Suitability samples (SST) by aliquoting 0.1 mL of it into labeled autosampler vials.
C. Dry each vial under nitrogen on a warm plate set at 30° C.
D. Add 100 μL of Sylon BFT (99:1 BSTFA:TMCS) to each vial. Cap tightly and swirl gently.
E. Derivatization takes place in a 90° C. oven for 1 hour.
F. Samples are run through the GC-FIDS and analyzed.

### Procedure for Syringe Filter Polymer Cleaning

1) materials used in syringe cleaning procedure:
A. 0.15% Oil Red O dyed >95% pure Glyceryl Trioleate oil (See below-Procedure for preparing 0.15% Oil Red O dyed Triolein oil (Glyceryl trioleate))
B. Syringe Filters: 30 mm, 5 μm Nylon syringe filters; (Thermo Fischer Scientific Co, Waltham, MA: Part #F2500-50)
C. 24 ml Norm-ject luer (slip tip) syringes Ref #4200-00V0 (These are silicone free syringes)
D. Isopropanol (IPA)
E. Polymer Solutions/mixtures—diluted with distilled water to the desired percentage (typically 0.5% for a polymer only solution and 10% for a full formulation unless otherwise noted in the examples).
F. Plastic drain tubing having a diameter to fit over the slip tip of the syringe filter tightly

### Procedure for Preparing 0.15% Oil Red O Dyed Triolein Oil (Glyceryl Trioleate)

### Materials:

Triolein oil (Glyceryl trioleate) Sources:
1. Sigma PCode: 102126986, Lot #BCBW9872, >97% purity, Store @2-8° C.
2. MP Biomedicals, LLC PCode: 103122, Lot #SR00405, >95% purity, Store @ 2-8° C.
   Oil Red O dye: Sigma PCode: 09755-25G, Lot #018K0669
   Appropriate size glass containers (4 oz or less)
   1.5 ml plastic Eppendorf centrifuge tubes
      10 ml Norm-ject luer (Luer lock) syringes Ref #4100-X00V0 (only substitute NON-silicone syringes)
      Syringe Filter: PALL Sciences Acrodisc 32 mm with 5 μm Supor Membrane, Product Code: 4650
Mettler Toledo XS1003S: 3-Place Balance (minimum)
Water bath capable of maintaining a temperature of 38 C±2 C

### Procedure:

1. Warm one or multiple samples of Glyceryl Trioleate to room temperature; combine multiple samples into the appropriate size glass container.
2. Weigh out the Oil Red O dye to a final concentration of 0.15% and add the dye to the Glyceryl trioleate.
3. Warm mixture to 38° C. in a water bath with repeated agitation/mixing for a maximum of 20 minutes after reaching the final temperature of 38° C.
4. Filter oil/dye mixture through a 5 μm syringe filter into an appropriate size glass container.
5. Dispense mixture into 1.5 ml size Eppendorf tube and store refrigerated @2-8° C.
   2) syringe pump:
Use syringe pump comparable to New Era Pump Systems Inc.
Model NE-100 Multi-Phaser
Follow manufactures instructions

### Syringe Pump Settings

Syringe Diameter: 20 mm (Note: The volume of liquid dispensed by the syringe is based on the diameter of the syringe and diameter setting must be changed if using different sizes of syringes).
Dispensing Volume set point: 5 mls
Cleaning Dispensing Rate: 5 mls/min
IPA Extraction Rate: 2.5 ml/min
3) controls
A. Instrumentation Operational Quality Control:

The daily Operational Quality Control for both the Syringe Pump and the Spectrophotometer should be performed and pass success criteria before either one can be used to generate data using this procedure. The performance check procedure for both instruments is listed below:

### Procedure:

1. Fill 24 ml syringe with water and place in syringe pump
2. Set syringe pump to a Rate of 5 ml/min
3. Place an empty tarred 50 ml tube directly under syringe tip to collect the dispensed water
4. Turn on pump and a start stopwatch
5. After pump finished dispensing stop the stopwatch and weigh the 50 ml tube
6. Repeat changing the rate from 5 ml/min to 2.5 ml/min

### Passing Criteria:

| Rate Setting | Weight (g) | Time (sec) |
|---|---|---|
| 5 ml/min | 4.9-5.1 | 58-62 |
| 2.5 ml/min | 4.9-5.1 | 58-62 |

B. Assay Controls:

### Control Description

| Control/Standard | Description |
|---|---|
| 0.5% Styleze CC-10 | High % reduction polymer control |
| EXP-19-CD0549-23 | |
| 0.5% Sorez HS 205 | Low % reduction polymer control |
| EXP-19-CD0549-24 | |
| IPA Extraction % Recovery | This control measures IPA extraction |
| | efficacy for removing dyed oil off the |
| | syringe filter |
| 100% Dye Tube Standard | This standard is used for calculating % |
| | reduction for test samples and % |
| | recovery for IPA Extraction control |

The three controls (Styleze CC-10, Sorez HS 205 and IPA Extraction Control) and the Dye Tube Standard listed in Table 1 should be run every time the syringe method is performed.
Note:
The weight of IPA used in the Dye Tube Standard should fall in the range of 3.7 g-3.9 g.

| | Control | | | | |
|---|---|---|---|---|---|
| | | | | | Dye Tube |
| | | | IPA | | Standard |
| | Styleze | Sorez | Extraction | Dye Tube | IPA |
| | CC-10 | HS 205 | Control | Standard | weight |
| | Results | | | | |
| | % | % | % | mg/AU | |
| | Reduction | Reduction | Recovery | (Absorb. Units) | grams |
| Mean | 66% | 30% | 84% | 50 | 3.8 |
| 2sd | 60%-72% | 18%-42% | 68%-100% | 46-54 | 3.7-3.9* |
| Range | | | | | |

Control Range Summary
*3.7-3.9 is not an 2sd range

C. Dye Tube Standard Preparation:

The Dye Tube Standard is used to calculate the % Reduction for the two controls and polymer test sample as well as the % Recovery for the IPA Extraction Control.
1. Pipette out between 48 mg-50 mg of dyed oil into a tarred 15 ml conical centrifuge tube, recording the weight.
2. Pipette between 3.7 g-3.9 g of IPA into the tube. Record weight 3. Vortex until all the dyed oil is solubilized in the IPA
D. IPA % Recovery Extraction Control:

Label 3 syringe filters as IPA extraction controls. Follow Step 1: Coating Filters in the Syringe Filter Wash Procedure.

Extract off dyed oil with IPA following Step 5: IPA Extraction in the Syringe Filter Wash Procedure.
4) Syringe filter wash procedure
A. Coating Filters

Label and dye coat all filters needed for the day's testing at the same time and use them on the same day.

TABLE 1
Describes the number of replicates used for the three Controls, the
Standard and Polymer test samples (polymer test samples are at
0.5% unless otherwise noted).

| 0.5% Styleze | 0.5% Sorez | IPA | | |
|---|---|---|---|---|
| CC-10 | HS 205 | Extraction | 100% Dye | Polymer |
| % Reduction | % Reduction | % Recovery | Tube | Test |
| Control | Control | Control | Standard | Samples |
| 1 Replicate | 1 Replicate | 3 Replicates | 4 Replicates | 3 Replicates/ |
| | | | | sample |

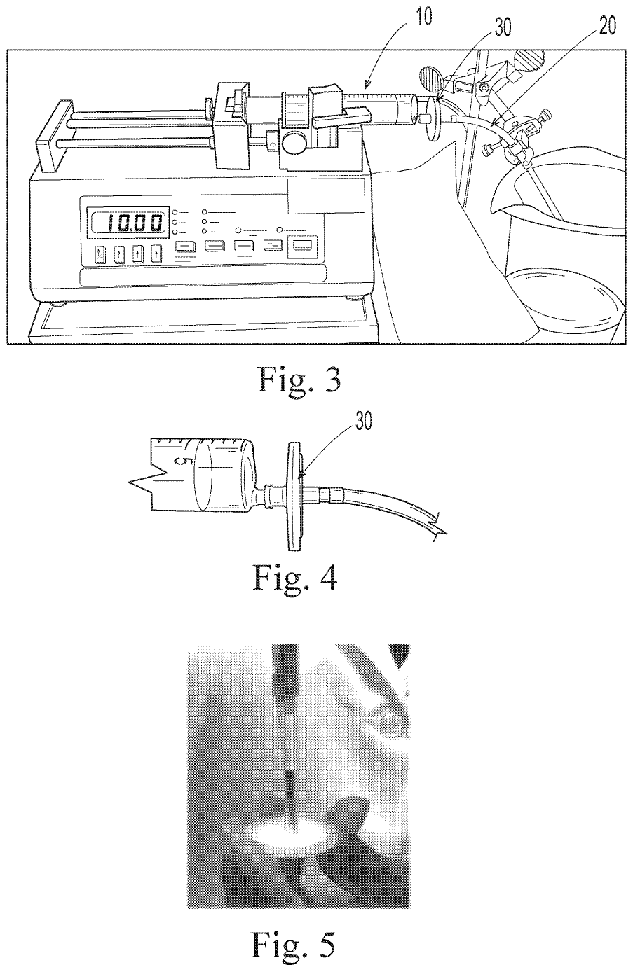

1. Coat the slip tip side of the syringe filter (see FIG. 5) using a micro pipettor with 48-50 mg of Oil O Red dyed Triolein oil by setting the micro-pipettor to a volume of ~58 ul to compensate for the lower density of the dyed oil.
2. Slowly fill the pipette tip with oil. Hold the filter level in one hand then lower the pipette tip just above the filter membrane then dispense the oil onto the filter.
3. Ensure the oil does not get hung up and reaches the nylon membrane.
Record the exact weigh of the coated syringe filter.
4. Keeping the filter level to the ground, allow enough time for the dyed oil to spread over the entire surface of the syringe filter (~20 minutes).
B. Polymer Wash
   1. Fill a new clean slip tip syringe with 20 mls of polymer solution.
   2. Place & secure the polymer containing syringe (10) into the syringe pump (see FIG. 3).
   3 Attach a drain tube (20) to the slip tip side (dyed side) of the filter (30) and direct the open-end of the tube into a waste container. Use a new/clean drain tube when testing a different polymer. (See FIGS. 3 and 4)
   4. Connect the luer lock side (see FIG. 4) of the oil coated filter (30) to the slip tip end of the syringe.
   5. Push the start button on the syringe pump and run 2 mls@(5 ml/min) of polymer solution through the filter then push the start button again to turn off pump. Soak for 2 minutes then push start button to pump the remaining 3mls through the filter.
C. Water Rinse
   1. Remove polymer syringe and attach a new clean syringe filled with 20 mls of reagent grade bottle or Milliq water and reattach the syringe filter.
   2. Push start button again and rinse filter with 5 mls@(5 ml/min) of water (no soak time).
D. Air Purge
   1. Remove water syringe and replace it with a clean dry syringe with the plunger pulled back to the 20 ml mark. Attach to the filter to the syringe and pump 10 ml@(5 ml/min) (2, 5 ml volumes) of air through the filter to purge the water out of the filter.

The IPA Extraction Step #5 is only completed when all the filters have completed above Steps 1-4.
E. Ipa extraction (change pump rate setting to 2.5 ml/min)
   1. Fill a new clean slip tip syringe with 20 mls of IPA and secure the syringe to the pump.
   2. Attach one end of a clean drain hose to the slip side of the syringe filter (Note: Use new drain hose for every filter extraction step) and the other end to the syringe. Tare a 15 ml tube on the balance then place the open end of the drain tube into the 15 ml centrifuge tube in order to collect the IPA and extracted dye off the syringe filter.
   3. Push the start button. Rotate the syringe filter 360 degrees during the entire 5 ml extraction period.
   4. After the 5 mls of IPA has passed through the filter, disconnect the filter from the syringe and attach a clean empty syringe with the plunger pulled back to the 20 ml line. Manually push 20 mls of air through the filter, collecting any IPA expelled out into the tube. Reweigh tube and record IPA extraction gram weight.
   5. Repeat steps 1~4 for each syringe test filters using a clean drain hose.
   6. Vigorously vortex each collection tube for 10-15 secs or longer until the dyed oil is completely dissolved in the IPA before reading sample in spectrophotometer
F. Sample Analysis

Spectrophotometer comparable to VWR UV-3100PC
1. Setup spectrophotometer following manufactures instructions.
2. Set wavelength of spectrophotometer back to 518 nm after performing spectrophotometer OQ and blank against IPA
3. Read and record absorbance
5) data & calculations

Manually determine the mg of dyed oil extracted off the filter using the following formula:

mg of extracted dyed oil=(Absorb of test filter/Absorb of Dye Tube Standard)×the average mg of dyed oil pipetted into the Dye Tube Standard

Percent Reduction=1−(mg of extracted dyed oil/mg of dyed oil applied to filter)×100

For determining polymer only properties, such as molecular weight, charge density, surface tension, and cleaning; a 2-5% polymer solution in distilled water was made at room temperature and then further diluted with distilled water as needed (and noted) for each method. To make the solution, an appropriately sized jar was tarred, the necessary amount of water and stir bar was added. The jar was placed on a stir plate and the necessary amount of stirring applied. The necessary amount of polymer was then added (if supplied as a powder then by weighing first, if provided as a solution in water already then by syringe after weighing making note of the active level of polymer in the starting solution). The solution was then left to mix overnight if needed for dissolution (typically for the naturally derived polymers). In cases where only a small sample amount was needed, the pre-weighed polymer was instead added to the pre-weighed water in a Wheaton vial or centrifuge tube. If needed a vortex mixer was then used to aid in dissolution.

For determining the properties of full formulations, such as viscosity or cleaning or for product making, the following procedure was followed to make a typical 2-5% polymer active formulation that was then diluted as necessary. Actual mass was recorded for each ingredient. In a container of sufficient size, the required amount of ambient temperature water was added. The polymer (powder or solution, keeping note of the starting active level of polymer) was slowly dispersed, while mixing, using an overhead mixer at approximately 320 rpm for around 10 minutes. Additional materials were then added, while mixing, including humectants, cosolvents, preservatives, scalp actives, opacifiers, sensates, feel actives, botanicals, vitamins, and sebum modifying actives. After the additional materials were added the speed was increased to approximately 415 rpm. If no surfactant was used, then the perfume was added to the main batch while continuing to mix. If a low level of surfactant was used, it was pre-mixed with the perfume before addition to the batch. To make the pre-mix, the perfume was added to the surfactant in an appropriate container. An overhead mixer (IKA RW 20 digital overhead mixer or similar) at a speed of approximately 150-200 rpm was used to make the pre-mix. Then add this pre-mix to the main batch while mixing. Citric acid was added to the main batch and the mixer speed increased to approximately 700 rpm. If aloe was used, it was added at this time while still mixing. If Styleze CC10 (or other very viscose polymer solution) was used, the mixer was stopped and the Styleze CC10 added; the mixer was then restarted and the speed slowly increased back to 700 rpm, with mixing continuing for 15 minutes.

SAMPLES A1-10, as shown in TABLE 2 below, illustrate inventive samples wherein synthetic or naturally derived cationic polymers meet the requirements of claim 1 (MW≥400,000, surface tension ≥45 mN/m, and CD between 0.4-4 meq/g) and still clean hair of sebum (SYRINGE FILTER POLYMER CLEANING PROCEDURE sebum removal ≥45%). Without being bound by theory it is believed that these formulations allow for the cationic polymer to be attracted to the negatively charged hair or skin surface and displace sebum. For SAMPLE A1, the values are for the non-preserved polymer (still called Styleze CC10). The preservative in Styleze CC10 is a known surfactant and reduces the surface tension of the polymer solution to below 45 mN/m. However, the non-preserved version with a surface tension of 70 mN/m still removes ≥45% of the sebum in the SYRINGE FILTER POLYMER CLEANING PROCEDURE. For the synthetic polymers in SAMPLE A1-A5 the SYRINGE FILTER POLYMER CLEANING PROCEDURE and other cleaning methods are done using a starting formulation at 5% polymer that is then diluted in the method prep to 0.5% in the SYRINGE FILTER POLYMER CLEANING PROCEDURE (to take into account the dilution that occurs in a shower setting). This dilution is not done in the PROCEDURE TO WASH SEBUMED HAIR SWITCH (since it occurs naturally during the washing of the hair switch in the sink). However for the naturally derived polymers SAMPLES A6-A10, their viscosity is often much higher, creating thicker formulas that are difficult to work with. As such the starting formulation is often 2% (and noted in TABLE 2) and then diluted in the method prep to 0.2% with distilled water for the SYRINGE FILTER POLYMER CLEANING PROCEDURE). Again this dilution is not done in the PROCEDURE TO WASH SEBUMED HAIR SWITCH (since it occurs naturally during the washing of the hair switch in the sink).

TABLE 2

| | | | | | % | % Sebum | % Sebum |
|---|---|---|---|---|---|---|---|
| | | | | | Starting | Removed | Removed |
| | | | Surface | Charge | Polymer | with Hair | with |
| Inventive | Commercial | | Tension | Density | Before | Switch | Syringe |
| SAMPLE | Polymer | MW (Da) | (mN/m) | (meq/g) | Dilution | Method | Method |
| A1 | Ashland | 1,200,000- | 70 | 1.5 | 5 | 85 | 66 |
| | Styleze | 1,500,000 | | | | | |
| | CC10 | | | | | | |
| A2 | Ashland | 2,700,000 | NT | 1.3 | 5 | 77 | 64 |
| | Styleze W10 | | | | | | |
| A3 | Ashland | 1,400,000 | 58 | 0.8 | 5 | 76 | 49 |
| | Conditioneze | | | | | | |
| | NT-20 | | | | | | |
| A4 | Ashland | 1,000,000 | 66 | 0.4 | 5 | 75 | 47 |
| | Gafquat | | | | | | |
| | 755N-O | | | | | | |
| A5 | BASF | 400,000 | 73 | 3.5 | 5 | 85 | 45 |
| | Luviquat | | | | | | |
| | HM552 | | | | | | |
| A6 | Dow UCare | 800,000- | 70 | 1.3 | 5 | 89 | 72 |
| | JR30M | 1,300,000 | | | | | |
| A6B | Dow UCare | 800,000- | 70 | 1.3 | 2 | NT | 63 |
| | JR30M | 1,300,000 | | | | | |
| A7 | BASF | 4,100,000 | 51 | 1.1 | 2 | 87 | 61 |
| | Dehyquart | | | | | | |
| | Guar HP | | | | | | |
| A8 | Dow UCare | NR | 71 | NR | 3 | 79 | 66 |
| | KG30M | | | | | | |
| A8B | Dow UCare | NR | 71 | NR | 2 | 77 | 57 |
| | KG30M | | | | | | |
| A9 | Dow Ucare | 1,300,000 | 70 | 1.1 | 5 | 76 | 72 |
| | LR30M | | | | | | |
| A9B | Dow UCare | 1,300,000 | 70 | 1.1 | 2 | NT | 55 |
| | LR30M | | | | | | |
| A10 | Dow UCare | 450,000 | 71 | 1.5 | 5 | 74 | 51 |
| | JR400 | | | | | | |
| A10B | Dow UCare | 450,000 | 71 | 1.5 | 2 | NT | 45 |
| | JR400 | | | | | | |

As shown in TABLE 3, SAMPLES B1-13 illustrate comparative samples wherein synthetic or naturally derived cationic polymers do not meet the requirements of claim 1 (MW≥400,000 and CD between 0.4-4 meq/g) and do not clean hair of sebum (SYRINGE FILTER POLYMER CLEANING PROCEDURE sebum removal <45%). As shown below in TABLE 3, without being bound by theory it is believed that these formulations do not have the necessary MW (size) or charge density to allow for the cationic polymer to be attracted to the negatively charged hair or skin surface and displace sebum. SAMPLES B1-B5 have too low a molecular weight. Without wishing to be bound to theory, it is believed they are too small in size to adequately cover the hair or skin surface to displace the sebum. SAMPLES B3-B5 also have too high a charge density. Without wishing to be bound by theory, it is believed that at such a high charge density they repel each other and are not adequately able to cover the hair or skin surface to displace the sebum. SAMPLES B6-B9 are nonionic. Without wishing to be bound by theory, it is believed they are not attracted to the negatively charged hair or skin surface. SAMPLES B10-B11 are anionic. Without wishing to be bound by theory, it is believed they are repelled by the negatively charged hair or skin surface.

TABLE 3

| | | | | | % Sebum | % Sebum |
|---|---|---|---|---|---|---|
| | | | | % Starting | Removed | Removed |
| | | | Charge | Polymer | with Hair | with |
| Comparative | Commercial | | Density | Before | Switch | Syringe |
| SAMPLE | Polymer | MW (Da) | (meq/g) | Dilution | Method | Method |
| B1 | BASF | 100,000 | 2.4 | 5 | NT | 35 |
| | Luviquat | | | | | |
| | FC370 | | | | | |
| B2 | BASF | 80,000 | 3.9 | 5 | NT | 36 |
| | Luviquat | | | | | |
| | FC550 | | | | | |
| B3 | Ashland N- | 300,000 | 5.3 | 5 | 66 | 33 |
| | DurHance | | | | | |
| | A1000 | | | | | |
| B4 | Lubrizol | 150,000 | 6.7 | 5 | NT | 44 |
| | Merquat 100 | | | | | |
| B5 | BASF | 40,000 | 7.9 | 5 | NT | 28 |
| | Luviquat | | | | | |
| | Excellence | | | | | |
| B6 | Ashland | 1,000,000 | 0.1 | 5 | 57 | 37 |
| | Copolymer | | | | | |
| | 845-O | | | | | |
| B7 | Ashland | 1,000,000 | 0.0 | 5 | 54 | 30 |
| | Sorez HS205 | | | | | |
| B8 | Dow | NT | 0.0 | 5 | NT | 33 |
| | Methocel E5 | | | | | |
| B9 | BASF | 24,000 | 0.0 | 5 | NT | 43 |
| | Sokalan HP22G | | | | | |
| B10 | Ashland | 1,000,000 | −4.4 | 5 | NT | 34 |
| | Styleze 2000 | | | | | |
| B11 | BASF | 12,000 | −4.1 | 5 | NT | 31 |
| | Sokalan CP9 | | | | | |

SAMPLES C1-C2 illustrate controls both positive (commercial shampoo) and negative (water) to show the upper and lower limits for the cleaning methods. As shown in TABLE 4, SAMPLE C1 (water) removes just 29% of the sebum in the SYRINGE FILTER POLYMER CLEANING PROCEDURE demonstrating that water alone is not enough to remove sebum from hair and skin. SAMPLE C2 Pantene Pro V Commercial Shampoo removes 89% of the sebum in the SYRINGE FILTER POLYMER CLEANING PROCEDURE demonstrating that traditional high surfactant level shampoos remove a majority of the sebum from hair and skin.

TABLE 4

| | | % | % Sebum | % Sebum |
|---|---|---|---|---|
| | | Starting | Removed | Removed |
| Comparative | | Active | with Hair | with |
| Control | | Before | Switch | Syringe |
| SAMPLE | Description | Dilution | Method | Method |
| C1 | Water | NA | 43 | 29 |
| C2 | Pantene Pro | NA | 98 | 89 |
| | V Shampoo | | | |

SAMPLES D1-D7 illustrate the requirement regarding polymer molecular weight. The polymers are a series made in-house where the comonomer levels are kept the same at a 45% DMAA to 55% MAPTAC ratio (with a theoretical charge density of 2.5 meq/g) in order to study the influence of molecular weight. The claim 1 cut-off is greater than or equal to 400,000. As shown below in TABLE 5, SAMPLES D1-D4 all meet this requirement and have a SYRINGE FILTER POLYMER CLEANING PROCEDURE sebum removal ≥45%. SAMPLE D5 is right below this MW cut-off and its cleaning is just at the cut-off for the SYRINGE FILTER POLYMER CLEANING PROCEDURE and is below the cut-off for the PROCEDURE TO WASH SEBUMED HAIR SWITCH. SAMPLES D6-D7 are both below the MW cut-off and the cleaning values are also low. Without being bound by theory, it is believed a minimum molecular weight is required in order to effectively coat the hair or skin and displace sebum. This is also shown in the commercial UCare series from DOW. Keeping the charge density constant, the molecular weight increases from JR125 to JR400 to JR30M and the percent sebum removed via the SYRINGE FILTER POLYMER CLEANING PROCEDURE also increases from JR125 (34%) to JR400 (45%) to JR30M (63%).

Polymers of the present invention, as illustrated by SAMPLES D and E, and shown below in TABLES 5 & 6 and 7 & 8 respectively, may be made by any suitable process known in the art. For example, the polymer may be made by radical polymerization.

MW was controlled via control of the reaction concentration, initiator concentration and chain transfer agent (isopropanol) concentration as well as reaction temperature.

Increasing the monomer concentration generally increases the molecular weight.

Increasing the initiator concentration generally decreases the molecular weight.

Increasing the chain transfer agent (isopropanol) concentration generally decreases the molecular weight.

Charge density, for these polymers is a measure of the amount (moles- or equivalents) of positive charge per mass of polymer.

For these polymers the charge results from the MAPTAC monomer and it's ammonium quat structure.

For a 45/55 DMAA/MAPTAC copolymer—the theoretical composition is 45 grams of DMAA (MW 99.13 g/mole) and 55 grams of MAPTAC (MW 220.74).

55 grams of MAPTAC is 0.249 moles or equivalents of charge and for this composition that is per 100 grams of materials—resulting in a calculated charge density of 0.249/100=0.00249 equivalent per gram or 2.49 milliequivalents/gram.

### Non-Limiting Synthesis Examples Sample Preparation

a. Poly (DMAA-MAPTAC)

To a 250 mL reaction vessel, a quantity of dimethylacrylamide (available from Sigma Aldrich, catalog #274135), methacryloylaminopropyl trimethylammonium chloride (MAPTAC) (50% active solution in water, available from Sigma Aldrich, catalog #280658), and an additional amount of water (available from VWR, catalog #BDH1168 were added. A chain transfer agent, isopropyl alcohol (available from VWR, catalog #PX1835-6) was added when required. An initiator solution comprised of 2,2′-azobis(2-methylpropionamidine) dihydrochloride [available from Sigma Aldrich, catalog #440914] dissolved in water was also added. The reaction vessel was sealed, sparged for 3 minutes under an inert gas such as nitrogen, and then heated to a temperature of 56° C. for a minimum of 24 hours. The resultant polymer solution was diluted to approximately 3% active with water to form a free-flowing fluid. This fluid was poured into a pan, and froze at −30 C, and freeze dried by vacuum evaporation. All monomer, initiator, and solvent amounts can be found in detail in TABLES 6 (for SAMPLES D1-7) and 8 (for SAMPLES E1-9). 1. Note, methacryloylaminopropyl trimethylammonium chloride is received as a 50% solution in water. The MAPTAC values in Tables 6 and 8 do not reflect the mass of the water. Instead, the water from the MAPTAC sample is included with the total mass of water.

TABLE 5

| | | | % | % Sebum | % Sebum |
|---|---|---|---|---|---|
| | | | Starting | Removed | Removed |
| | | | Polymer | with Hair | with |
| | | | Before | Switch | Syringe |
| SAMPLE | Polymer | MW (Da) | Dilution | Method | Method |
| D1 | 45% | 1,600,000 | 5 | 73 | 60 |
| | DMAA-55% | | | | |
| | MAPTAC | | | | |
| D2 | 45% | 1,100,000 | 5 | NT | 62 |
| | DMAA-55% | | | | |
| | MAPTAC | | | | |
| D3 | 45% | 970,000 | 5 | NT | 65 |
| | DMAA-55% | | | | |
| | MAPTAC | | | | |
| D4 | 45% | 400,000 | 5 | NT | 50 |
| | DMAA-55% | | | | |
| | MAPTAC | | | | |
| D5 | 45% | 354,000 | 5 | 56 | 47 |
| | DMAA-55% | | | | |
| | MAPTAC | | | | |
| D6 | 45% | 216,000 | 5 | 53 | 40 |
| | DMAA-55% | | | | |
| | MAPTAC | | | | |
| D7 | 45% | 114,000 | 5 | 50 | 32 |
| | DMAA-55% | | | | |
| | MAPTAC | | | | |

TABLE 6

| | | | | MAPTAC | Water | Initiator | |
|---|---|---|---|---|---|---|---|
| SAMPLE | Polymer | MW (Da) | DMAA (g) | (g) | (g) | (g) | IPA (g) |
| D1 | 45% DMAA- | 1,600,000 | 9 | 11 | 80 | 0.2 | 0 |
| | 55% MAPTAC | | | | | | |
| D2 | 45% DMAA- | 1,100,000 | 9 | 11 | 113 | 0.2 | 0 |
| | 55% MAPTAC | | | | | | |
| D3 | 45% DMAA- | 970,000 | 9 | 11 | 180 | 0.2 | 0 |
| | 55% MAPTAC | | | | | | |
| D4 | 45% DMAA- | 400,000 | 9 | 11 | 178 | 0.2 | 2 |
| | 55% MAPTAC | | | | | | |
| D5 | 45% DMAA- | 354,000 | 4.5 | 5.5 | 86 | 0.2 | 5 |
| | 55% MAPTAC | | | | | | |
| D6 | 45% DMAA- | 216,000 | 4.5 | 5.5 | 81 | 0.2 | 9 |
| | 55% MAPTAC | | | | | | |
| D7 | 45% DMAA- | 114,000 | 4.5 | 5.5 | 72 | 0.2 | 18 |
| | 55% MAPTAC | | | | | | |

SAMPLES E1-E9 illustrate the requirement regarding charge density, as shown below in TABLE 7. The polymers are a series made in-house where the comonomer levels are systematically varied (while attempting to keep the molecular weight relatively the same) in order to study the influence of charge density. The claim 1 requirement is for a charge density between 0.4 and 4 meq/g. There is a general increase followed by a decrease in sebum removal as the charge density is increased. Without being bound by theory, it is believed that a minimum charge density is required to attract the polymer to the negatively charged skin or hair surface but that too high a charge density can cause repulsion of the cationic polymer to itself and prevent effective levels to be deposited and displace the sebum. SAMPLES E1-E8 all meet the charge density requirement, and all have high levels of sebum removal in the SYRINGE FILTER POLYMER CLEANING PROCEDURE. SAMPLE E9 has a higher charge density and is at the cut-off for the SYRINGE FILTER POLYMER CLEANING PROCEDURE and has a very low sebum removal by the PROCEDURE TO WASH SEBUMED HAIR SWITCH.

TABLE 7

| | | | | % | % Sebum | % Sebum |
|---|---|---|---|---|---|---|
| | | | Theoretical | Starting | Removed | Removed |
| | | | Charge | Polymer | with Hair | with |
| | | | Density | Before | Switch | Syringe |
| SAMPLE | Polymer | MW (Da) | (meq/g) | Dilution | Method | Method |
| E1 | 87% DMAA- | 3,300,000 | 0.6 | 5 | 65 | 63 |
| | 13% MAPTAC | | | | | |
| E2 | 77% DMAA- | 3,000,000 | 1.0 | 5 | 70 | 65 |
| | 23% MAPTAC | | | | | |
| E3 | 67% DMAA- | 5,000,000 | 1.5 | 5 | NT | 64 |
| | 33% MAPTAC | | | | | |
| E4 | 65% DMAA- | 1,300,000 | 1.6 | 5 | NT | 55 |
| | 35% MAPTAC | | | | | |
| E5 | 56% DMAA- | 6,400,000 | 2.0 | 5 | NT | 75 |
| | 44% MAPTAC | | | | | |
| E6 | 45% DMAA- | 1,600,000 | 2.5 | 5 | 73 | 60 |
| | 55% MATPAC | | | | | |
| E7 | 26% DMAA- | 1,100,000 | 3.4 | 5 | 62 | 57 |
| | 74% MAPTAC | | | | | |
| E8 | 11% DMAA- | 1,500,000 | 4.0 | 5 | NT | 59 |
| | 89% MAPTAC | | | | | |
| E9 | 100% MAPTAC | 932,000 | 4.5 | 5 | 47 | 48 |

TABLE 8

| | | | | MAPTAC | | Initiator |
|---|---|---|---|---|---|---|
| SAMPLE | Polymer | MW (Da) | DMAA (g) | (g) | Water (g) | (g) |
| E1 | 87% DMAA- | 3,300,000 | 17.4 | 2.6 | 80 | 0.2 |
| | 13% MAPTAC | | | | | |
| E2 | 77% DMAA- | 3,000,000 | 15.4 | 4.6 | 80 | 0.2 |
| | 23% MAPTAC | | | | | |
| E3 | 67% DMAA- | 5,000,000 | 13.4 | 6.6 | 80 | 0.1 |
| | 33% MAPTAC | | | | | |
| E4 | 65% DMAA- | 1,300,000 | 13 | 7 | 80 | 0.2 |
| | 35% MAPTAC | | | | | |
| E5 | 56% DMAA- | 6,400,000 | 11.2 | 8.8 | 80 | 0.1 |
| | 44% MAPTAC | | | | | |
| E6 | 45% DMAA- | 1,600,000 | 9 | 11 | 80 | 0.2 |
| | 55% MAPTAC | | | | | |
| E7 | 26% DMAA- | 1,100,000 | 5.2 | 14.8 | 80 | 0.2 |
| | 74% MAPTAC | | | | | |
| E8 | 11% DMAA- | 1,500,000 | 2.2 | 17.8 | 80 | 0.1 |
| | 89% MAPTAC | | | | | |
| E9 | 100% MAPTAC | 932,000 | 0 | 20 | 80 | 0.2 |

SAMPLES F1-F11, as shown below in TABLE 9, illustrate the requirement regarding level of cationic polymer. The claim 1 requirement is for a cationic polymer level of 1-10%. Without being bound by theory, at levels less than 1% similar to that observed in commercial conditioners it is believed that not enough polymer is deposited onto the hair or skin surface to displace the sebum. At levels higher than 10% of these high cationic polymers there are issues with dissolution and too high a viscosity for ease of consumer dispensing and spreading during use. SAMPLES F1-F5 and F6-F8 demonstrate the decrease in cleaning performance with decreasing polymer level with a cut-off around 1%. F9-F11 demonstrate that high levels of a very high molecular weight, high viscosity formula like Dehyquart Guar HP can result in lower cleaning performance in the PROCEDURE TO WASH SEBUMED HAIR SWITCH as it becomes increasing difficult to get the polymer in solution (in fact F9 is not a solution but a gel) and to spread the polymer over the hair for effective cleaning.

TABLE 9

| | | | | | % Sebum | % Sebum |
|---|---|---|---|---|---|---|
| | | | | % Starting | Removed | Removed |
| | | | Charge | Polymer | with Hair | with |
| | Commercial | | Density | Before | Switch | Syringe |
| SAMPLE | Polymer | MW (Da) | (meq/g) | Dilution | Method | Method |
| F1 | Ashland | 1,200,000- | 1.4 | 5 | 85 | 66 |
| | Styleze CC10 | 1,500,000 | | | | |
| F2 | Ashland | 1,200,000- | 1.4 | 3 | 83 | 62 |
| | Styleze CC10 | 1,500,000 | | | | |
| F3 | Ashland | 1,200,000- | 1.4 | 1 | 65 | 54 |
| | Styleze CC10 | 1,500,000 | | | | |
| F4 | Ashland | 1,200,000- | 1.4 | 0.5 | 49 | 49 |
| | Styleze CC10 | 1,500,000 | | | | |
| F5 | Ashland | 1,200,000- | 1.4 | 0.05 | 45 | 42 |
| | Styleze CC10 | 1,500,000 | | | | |
| F6 | Dow UCare | NR | NR | 3 | 79 | 66 |
| | KG30M | | | | | |
| F7 | Dow UCare | NR | NR | 2 | 77 | 61 |
| | KG30M | | | | | |
| F8 | Dow UCare | NR | NR | 1 | 76 | 47 |
| | KG30M | | | | | |
| F9 | BASF Dehyquart | 4,100,000 | 1.1 | 3 | 68 | NT |
| | Guar HP | | | | | |
| F10 | BASF Dehyquart | 4,100,000 | 1.1 | 2 | 87 | 56 |
| | Guar HP | | | | | |
| F11 | BASF Dehyquart | 4,100,000 | 1.1 | 1 | 81 | NT |
| | Guar HP | | | | | |

SAMPLES G1-G10, as shown in TABLE 10, are comparative SAMPLEs and illustrate that previously disclosed so called, “cationic polymers” do not meet the claim 1 requirement of a surface tension greater than or equal to 45 mN/m. As such in this filing they would be characterized as surfactants and although they might provide a minimum level of cleaning based on the SYRINGE FILTER POLYMER CLEANING PROCEDURE, they are not surfactant free nor low surfactant formulations.

TABLE 10

| | | | % | % Sebum | % Sebum |
|---|---|---|---|---|---|
| | | | Starting | Removed | Removed |
| | | Surface | Polymer | with Hair | with |
| | Commercial | Tension | Before | Switch | Syringe |
| SAMPLE | Polymer | (mN/m) | Dilution | Method | Method |
| G1 | Croda | 42 | 5 | 42 | 44 |
| | Mirustyle CP | | | | |
| G2 | BASF | 37 | 5 | 50 | 54 |
| | Lamequat L | | | | |
| G3 | Crodacel | 37 | 5 | 59 | 49 |
| | QM PE-LQ | | | | |
| G4 | Promois | 37 | 5 | 47 | 51 |
| | WK-HCAQ | | | | |
| G5 | Promois WS- | 36 | 5 | 44 | 32 |
| | HCAQ | | | | |
| G6 | Promois | 36 | 5 | NT | 37 |
| | WG-CAQ | | | | |
| G7 | Promois | 35 | 5 | NT | 37 |
| | WK-SAQ | | | | |
| G8 | Promois | 37 | 5 | NT | 45 |
| | S-CAQ | | | | |
| G9 | Crodacel | 37 | 4% | 68 | 53 |
| | QM | | Crodacel + | | |
| | Lamequat L | | 3% | | |
| | | | Lamequat | | |
| G10 | Crodacel | 37 | 2% | 49 | 53 |
| | QM | | Crodacel + | | |
| | Lamequat L | | 2.25% | | |
| | | | Lamequat | | |

SAMPLES H1-H4, as shown below in TABLE 11, are inventive full formulations that met claim 1 requirements and provide the desired level of cleaning via the SYRINGE FILTER POLYMER CLEANING PROCEDURE.

TABLE 11

| | | % Sebum | % Sebum |
|---|---|---|---|
| | | Removed | Removed |
| | | with Hair | with |
| | | Switch | Syringe |
| SAMPLE | Formulation | Method | Method |
| H1 | Q.S. Water, 5% Styleze CC10, 3% | 89 | 61 |
| | Glycerin, 1% Methocel E50, 0.55% | | |
| | Sodium Citrate, 0.5% Potassium | | |
| | Sorbate, 0.12% Citric Acid | | |
| H2 | Q.S. Water, 3% UCare KG30M, 3% | 87 | 63 |
| | Glycerin, 0.55% Sodium Citrate, 0.5% | | |
| | Potassium Sorbate, 0.12% Citric Acid | | |
| H3 | Q.S. Water, 3% UCare KG30M, 3% | 88 | NT |
| | Glycerin, 0.55% Sodium Citrate, 0.5% | | |
| | Potassium Sorbate, 0.12% Citric Acid, | | |
| | 0.04% Fragrance | | |
| H4 | Q.S. Water, 3% Glycerin, 2% | NT | 62 |
| | Dehyquart Guar HP, 0.6% Fragrance, | | |
| | 0.55% Sodium Citrate, 0.5% | | |
| | Potassium Sorbate, 0.1% Citric Acid, | | |
| | 0.03% Aloe | | |

### Example 2

As shown in TABLE 12, SAMPLES 11-19 were tested for irritancy/gentleness using the TRPA1 CELL CULTURE METHOD, TRPV1/V3 CELL CULTURE METHOD, TRPM8 CELL CULTURE METHOD, with SAMPLES 11-13 representing formulations of the present invention and SAMPLES 14-19 comparative commercially available Shampoo Formulations. In embodiments, compositions of the present invention at a concentration of 4000 ppm or higher, have a level of TRPA1 V1, V3, or M8 Receptor activation that is <100 AUC, preferably <50 AUC, as determined by the TRPA1, V1, V3, or M8 Cell Culture Method respectively.

### TRPA1 Cell Culture Method

In order to determine whether TRPA1 is activated, the intracellular calcium ion (Ca²⁺) level from transfected cells with the TRPA1 receptor gene was measured. HEK-293 cells stably transfected with human TRPA1 were grown in 15 ml growth medium [high glucose DMEM (Dulbecco's Modification of Eagle's Medium) supplemented with 10% FBS (fetal bovine serum), 100 ug/ml Penicillin/streptomycin, 100 μg/ml G418] in a 75 Cm² flask for 3 days at 37° C. in a mammalian cell culture incubator set at 5% CO₂ and 95% humidity. Cells were detached with addition of 10 ml of PBS (phosphate buffered saline) without calcium or magnesium by hand shaking gently and transferred to a 50 ml tube and centrifuged at 850 rpm for 3 minutes to remove PBS. After centrifugation, a pellet of cells was formed in the bottom of the tube separating them from the supernatant solution. The supernatant was discarded and the cell pellet suspended in 1 ml of fresh growth medium to which 5 μl (12.5 μg) of Fluo-4 AM (Invitrogen) calcium indicator was added and incubated for 60 minutes with gentle shaking. Fluo-4 AM is a fluorescent dye used for quantifying cellular Ca²⁺ concentrations in the 100 nM to 1 μM range. At the end of the 60 minutes, 45 ml of assay buffer [1×HBSS (Hank's Balanced Salt Solution), 20 mM HEPES (4-(2-Hydroxyethyl)-1-piperazineethanesulfonic acid)] was added to wash the cells and the resulting combination was then centrifuged at 850 rpm for 3 minutes to remove excess buffer and Fluo-4 AM calcium indicator.

The pelleted cells were re-suspended in 10 ml assay buffer and 90 μl aliquots (~50,000 cells) per well delivered to a 96-well assay plate containing 10 μl of test compounds (1 mM in assay buffer, final concentration 100 μM) or buffer control and incubated at room temperature for 20 minutes. After 20 minutes, the plate was placed into a fluorometric imaging plate reader (FLIPR Tetra from Molecular Devices) and basal fluorescence recorded (excitation wavelength 494 nm and emission wavelength 516 nm). Then 20 μl of the TRPA1 agonist (50 μM AITC at final concentration) was added and fluorescence recorded. For determining the direct effect of test compounds on TRPA1, fluorescence was measured immediately after addition of each compound.

### TRPV1/V3 Cell Culture Method

In order to determine whether TRPV1 or TRPV3 is activated, the intracellular calcium ion (Ca²⁺) level from transfected cells with the TRPV1 or TRPV3 receptor gene was measured. For regular cell maintenance, TRPV1 or TRPV3-expressing cells were grown in in high glucose DMEM (Dulbecco's Modification of Eagle's Medium) supplemented with 10% FBS (fetal bovine serum), 100 μg/ml Penicillin/streptomycin, and 100 μg/ml G418 in a 75 Cm² flask for 3 days at 33° C. for TRPV1 and 37° C. for TRPV3 in a mammalian cell culture incubator set at 5% CO₂ and 95% humidity. TRPV1 or TRPV3 cells were detached by treating flasks with 10 ml of Phosphate Buffered Saline (PBS), without calcium or magnesium. Detached cells from the five flasks were combined in a 50-ml conical tube and centrifuged at low speed (800-900 rpm) for 3 minutes. Gently removed the supernatant. Re-suspend the cell pellet in 4 ml of growth medium. 50 μg of Fluo-4 AM calcium dye (Invitrogen) was dissolved in 20 μl of Pluronic F-127 (20% solution in DMSO); this solution was then added to cell suspension for 60 minutes, with gentle shaking, at room temperature.

The cells were centrifuged again at low speed (800-900 rpm) for 3 minutes. The cells were then washed once with 45 ml of assay buffer (1×HBSS, 20 mM HEPES), and pelleted again by centrifuging at low speed (800-900 rpm) for 3 minutes. Re-suspended the cells in assay buffer and calculate number of cells. Following this, diluted the cells to a volume of assay buffer, such that ~50,000 cells were dispensed in 100 μl/well of a 96-well plate [BD Falcon micro test assay plate #353948].

The cells were incubated for 20 minutes at room temperature. Read the plates in the FLIPR instrument at excitation wavelength of 494 nm, and emission wavelength of 516 nm, to record baseline fluorescence. Next, added the assay buffer for negative control; specific agonist for positive control—350 nM capsaicin for TRPV1, and 2 μM ionomycin for general control, and 50 μM 2-APB (2-Aminoethoxydiphenyl borate) for TRPV3 and test materials to the wells, using the dispenser provided with the FLIPR machine. Recorded data at 1 second intervals at the first 100 seconds and then 10 second intervals. The collected data was then analyzed based on the value at 90 sec, max (peak) and area under the curve (AUC, total) for 10 min. This represented the direct effect of the test materials being added to TRPV1 or TRPV3 cells. The specificity was established by: Comparing the results with pCDNA3-control cells, dye control, and other TRP receptor cells, following similar protocols as above. Also, addition after preincubation for 10 min with Capsezapine (10 μM).

### TRPM8 Cell Culture Method

In order to determine whether TRPM8 is activated, the intracellular calcium ion (Ca²⁺) level from transfected cells with the TRPM8 receptor gene was measured. For regular cell maintenance, TRPM8-expressing cells were grown in high glucose DMEM (Dulbecco's Modification of Eagle's Medium) supplemented with 10% FBS (fetal bovine serum), 100 ug/ml Penicillin/streptomycin, 5 μg/ml blasticidin, and 100 μg/ml zeocin in a 75 Cm² flask for 3 days at 37° C. in a mammalian cell culture incubator set at 5% CO₂ and 95% humidity. TRPM8 expression was induced by addition of 100 ng/ml doxycycline overnight. TRPM8 cells (from 75 cm² flasks) were detached by treating flasks with 10 ml of Phosphate Buffered Saline (PBS), without calcium or magnesium. Detached cells from the five flasks were combined in a 50-ml conical tube and centrifuged at low speed (800-900 rpm) for 3 minutes. Gently removed the supernatant. Re-suspend the cell pellet in 4 ml of growth medium. 50 μg of Fluo-4 AM calcium dye (Invitrogen) was dissolved in 20 μl of Pluronic F-127 (20% solution in DMSO); this solution was then added to cell suspension for 60 minutes, with gentle shaking, at room temperature.

The cells were centrifuged again at low speed (800-900 rpm) for 3 minutes. The cells were then washed once with 45 ml of assay buffer (1×HBSS, 20 mM HEPES), and pelleted again by centrifuging at low speed (800-900 rpm) for 3 minutes. Re-suspended the cells in assay buffer and calculated the number of cells. Following this, diluted the cells to a volume of assay buffer, such that 55,000 cells were dispensed in 90 μl/well of a 96-well plate [BD Falcon micro test assay plate #353948]. The cells were incubated for 20 minutes at room temperature.

Read the plates in the FLIPR instrument at excitation wavelength of 494 nm, and emission wavelength of 516 nm, to record baseline fluorescence. Next, added assay buffer for negative control; specific agonist for positive control—30 uM WS-5 and 1 uM ionomycinand test materials to the wells, using the dispenser provided with the FLIPR machine. Recorded data at 1 second intervals at the first 100 seconds and at 10 second intervals. The collected data was then analyzed based on the value at 90 sec, max (peak) and area under the curve (AUC, total) for 10 min. This represented the direct effect of the test materials being added to TRPM8 cells. The specificity was established by: Comparing the results with pCDNA3-control cells, dye control and other TRP receptor cells, following similar protocols as above.

TABLE 12

| | | | TRPA1 | TRPV1 | TRPV3 | TRPM8 |
|---|---|---|---|---|---|---|
| | | Dose | (Direct | (Direct | (Direct | (Direct |
| SAMPLE | Description | (ppm) | AUC %) | AUC %) | AUC %) | AUC %) |
| I1 | 2% Dehyquart Guar HP | 4166 | −11 | −7 | −13 | −11 |
| I2 | 2% Dehyquart Guar HP + | 4166 | 4 | −1 | −9 | −2 |
| | 0.12% Citric Acid | | | | | |
| I3 | 2% Dehyquart Guar HP + | 4166 | 60 | 14 | 1 | 21 |
| | 1.1% perfume | | | | | |
| I4 | Aveno Pure Renewal Sulfate | 4166 | 232 | 225 | 226 | 235 |
| | Free Shampoo | | | | | |
| I5 | Free & Clear Shampoo | 4166 | 177 | 159 | 203 | 158 |
| I6 | Johnson & Johnson | 4166 | 172 | 175 | 126 | 155 |
| | Baby Shampoo | | | | | |
| I7 | L'Oreal Ever Pure Sulfate | 4166 | 163 | 146 | 151 | 166 |
| | Free Shampoo | | | | | |
| I8 | Dove Daily Moisture Shampoo | 4166 | 183 | 126 | 151 | 219 |
| I9 | L'Oreal Elvive Shampoo | 4166 | 185 | 198 | 213 | 170 |

In regard to TABLE 12, in general receptor activation values <50 mean the receptor wasn't activated, between 50-100 slightly activated, and >100 activated. TRPM8 receptor activation is associated with pain from cold, TRPA1 is associated with pain from extreme cold, TRPV3 is associated with pain from warming, and TRPV1 is associated with pain from hot as well as inflammation. SAMPLES I1-3 demonstrate the low TRPA1, TRPV1, TRPV3, TRPM8 responses even at doses of 4000 ppm or higher for formulations described in this patent. SAMPLES I4-9 are comparative SAMPLEs of commercial shampoos that are often described in the literature as gentler but which at doses of ~4000 ppm have a TRPA1, TRPV1, TRPV3, TRPM8 response greater than 100 AUC, demonstrating the SAMPLES of the present invention I1-I3 were “gentle” compared to commercial sensitive options of SAMPLES I4-I9.

### Example 3

Polymers of interest were tested for interfacial tension (as a 0.5% solution in water), as described in the TEST METHODS Section (INTERFACIAL TENSION MEASUREMENT). As shown previously in TABLES for the polymers of interest as surface tension, the surface tension of the polymers of interest do not dramatically reduce the surface tension of water indicating that these polymers do not meet the traditional definition of a surfactant. The interfacial tension data shows that these same polymers do reduce the tension of oil (mineral, olive, and castor) indicating that the polymers are surprisingly able to accommodate oils/perfumes in the full formulation.

TABLE 13

| IFT (mJ/m2) | Mineral Oil | Std Dev | Olive Oil | Std Dev | Castor Oil | Std Dev |
|---|---|---|---|---|---|---|
| Water | 47 | | 20.4 | | 15.43 | |
| Guar HP | 17.45 | 2.23 | NT | NT | 5.46 | 0.33 |
| KG30M | 38.37 | 0.23 | 17.11 | 0.07 | NT | NT |
| JR30M | 33.72 | 0.3 | 16.32 | 0 | NT | NT |
| JR400 | NT | NT | 16.41 | 0.03 | NT | NT |
| LR30M | 26.71 | 1.15 | 15.88 | 0.05 | NT | NT |
| Styleze W10 | NT | NT | 14.96 | 1.05 | NT | NT |
| N-Hance CG17 Guar | NT | NT | 14.36 | 0.04 | NT | NT |
| Clearhance C | NT | NT | 16.67 | 0.55 | NT | NT |

Guar HP = Sample A7 = BASF Dehyquart Guar HP
KG30M = Sample A8 = Dow Ucare KG30M
JR30M = Sample A6 = Dow Ucare JR30M
JR400 = Sample A10 = Dow Ucare JR400
LR30M = Sample A9 = Dow Ucare LR 30M
Styleze W10 = Sample A2 = Ashland Styleze W10
n-Hance = Ashland, INCI = guar hydroxypropyltrimonium chloride
Clearhance = Ashland, INCI = Cassia hydroxypropyltrimonium chloride

TEWL (transepidermal water loss) is a common clinical measure used to compare the mildness of surfactant-based formulations for skin (Rogiers 2001; Berardesca and Maibach 1990; Brink et al. 2019; O'Connor, Ogle, and Odio 2016; Thune et al. 1988). TEWL is considered to provide a relative understanding of individual skin barrier quality in relation to onset of disease, environmental impacts and exposure to topical formulations, such as consumer products (Alexander et al. 2018). Reduction of the skin's production of ceramides and barrier molecules have been correlated to increased measures of TEWL which are associated with poorer skin barrier quality and function and can be associated with visual degradation of skin in the form of increased dryness and erythema. Therefore, it is anticipated that changes in TEWL can provide insights for understanding the potential mildness of consumer formulations when applied to pre-clinical models which recapitulate aspects of the human skin barrier, such as organotypic epidermis models.

### Sample Prep and Test Method

Keratinocytes from human donors (available from LifeLine, Maryland) were cultivated with Complete Dermalife media until they reached 70-80% confluency. The keratinocytes were then subcultured per manufacturer's recommendations and used at either passage 1 or 2. For growth of keratinocytes on de-epidermized dermis (DED), two media were used. Medium 1 was used for the first three days while the cultures remained submerged and Medium 2 was used when cultures were raised to the air-liquid interface and then until the time of collection.

Medium 1 consists of: Dulbecco's Modified Eagle Medium (DMEM) and Ham's F-12 Nutrient Mixture at a ratio of 3:1, followed by the addition of Hyclone Cosmic Calf Serum (5%), Hydrocortisone (0.4 μg/ml), epidermal growth factor (0.02 mg/ml), transferrin (3 mg/ml), insulin (5 μg/ml), cholera toxin (0.02 μg/ml), triiodothyronine (2×10⁻¹¹ M), adenine (0.18 mM), sodium pyruvate 1×, GlutaMax 1× (Invitrogen), CaCl₂) (300 μM), 1×CD lipid concentrate 300 μM, fibroblast growth factor 7 (FGF-7) (10 ng/ml), and penicillin/streptomycin 1×.

Medium 2 consists of: medium 1 modified with the addition of 1% serum and removal of FGF-7 and 1 mM CaCl₂. Medium 1 was used for two days while the cultures remain submerged and Medium 2 was used for cultures raised to the air-liquid interface.

De-epithelialized dermis (DED) was prepared by removing fat from the skin sample with a scalpel, cutting the skin into squares measuring 1.25 cm², and placing the samples in 1M NaCl plus 10× penicillin/streptomycin. The sample was incubated overnight at 37° C. The following day, the epidermis was carefully peeled off with forceps and dermal tissue was stored in phosphate-buffered saline (PBS) plus 2× penicillin/streptomycin at 4° C. until ready for use.

Approximately 5×10⁵ keratinocytes in 50 μl of Medium 1 were pipetted into 10 mm cloning cylinders placed atop DEDs. 2 ml of Medium 1 was added to the bottom of the 6-well plate containing the transwell. The plates were incubated overnight at 33° C. in 5% CO₂ and 55% RH. The following day, the cloning cylinders were removed and cultures were submerged in Medium 1. At three days, cultures were raised to the air-liquid interface in Medium 2.

At day 7 at the air-liquid interface, the cultures were treated topically with full or diluted formulas by cotton swab or by pipetting 6-50 ul, depending on product viscosity. Treatments remain on the cultures for 20 minutes, and then were washed with 8 ml of water, patted dry with a cotton swab and returned to the incubator for 24 hours. Cultures were removed from incubator and place with lids open on the bench at room temperature for minimum of 20 minutes to equilibrate. Once equilibrated the cultures were placed on the lid of a sterile 150 mm petri dish and TEWL readings were taken using a Delfin Vapometer containing a silicone O-ring adaptor provided by the manufacturer.

TABLE 14

| | | Connecting |
|---|---|---|
| Sample | Mean | Letters |
| SLS (10%) | 26.03 | A |
| Gillette Fusion Proglide | 23.78 | A |
| King C Gillette | 20.9 | B |
| Pure by Gillette | 20.68 | B |
| 1% Styleze CC10 + 2% Dehyquart Guar HP + | 13.38 | CD |
| Preservative + Acid | | |
| 2% Dehyquart Guar HP + 1% Avocado Oil + | 12.38 | CDE |
| 0.6% Fragrance + Preservative + Acid | | |
| 2% Dehyquart Guar HP + Preservative + Acid | 12.15 | CDE |
| Water | 12.13 | CDE |
| 2% Dehyquart Guar HP + 1% Avocado Oil + | 11.63 | DE |
| Preservative + Acid | | |
| 2% Dehyquart HP + 1% Avocado Oil + 0.35% | 11.5 | DE |
| Fragrance + Preservative + Acid | | |
| Untreated | 10.53 | E |

* Samples not connected by the same letter are significantly different

The higher the value the worse the performance, as the value is a measure of the amount of water loss, which as described above is an indication of how much skin damage was done by the formulation.

### Example 4

### Oil Stability—all Testing was Done Visually (Appearance, Lack of Separation).

It's surprisingly unobvious that the present invention can get skin active oils and/or perfumes stabilized in formulations with little to no surfactant. The polymers act as solubilizers as shown by the interfacial tension values with oil, but they don't act as true surfactants as observed with the non-impacted surface tension with water, which is a further benefit to these novel polymers—they can clean (albeit by a different mechanism than surfactants) and they can provide some stability/solubility to the formulation for desired oils.

Two sets of data were prepared. The first set was for 1% of various skin care actives. The oils and the water soluble active (niacinamide) are all compatible under RT and accelerated (40° C.) conditions when checked visually for appearance and separation at initial, 2, 4, and 8 weeks. The two solid waxes (cocoa butter and shea butter) both failed at the initial check. The starting formula was 2% Dehyquart Guar HP+3% glcyerin+1.2% preservatives+0.8% buffer+0.03% aloe+1% skin active.

TABLE 15

| Skin Care | | RT | | | | 40° C. | | | |
|---|---|---|---|---|---|---|---|---|---|
| Active | | | 2 | 4 | 8 | | 2 | 4 | 8 |
| (1%) | | Initial | weeks | weeks | weeks | Initial | weeks | weeks | weeks |
| Avocado Oil | Liquids/Oils | Stable | Stable | Stable | Stable | Stable | Stable | Stable | Stable |
| Grapeseed Oil | | Stable | Stable | Stable | Stable | Stable | Stable | Stable | Stable |
| Capric Caprylic | | Stable | Stable | Stable | Stable | Stable | Stable | Stable | Stable |
| Triglyceride | | | | | | | | | |
| Acai Oil | | Stable | Stable | Stable | Stable | Stable | Stable | Stable | Stable |
| Buriti Oil | | Stable | Stable | Stable | Stable | Stable | Stable | Stable | Stable |
| Pataua Oil | | Stable | Stable | Stable | Stable | Stable | Stable | Stable | Stable |
| Niacinamide | Water Soluble | Stable | Stable | Stable | Stable | Stable | Stable | Stable | Stable |
| | Additives | | | | | | | | |
| Cocoa Butter | Solids/ | Fail - | NT | NT | NT | NT | NT | NT | NT |
| | Waxes | precipitate | | | | | | | |
| Shea Butter | | Fail - | NT | NT | NT | NT | NT | NT | NT |
| | | precipitate | | | | | | | |

NT—Not Tested

The second set was for 0.6% perfume. All of the perfumes were stable under 5° C., 25° C., and 46° C. when checked visually for appearance and separation at initial, 2, and 6 weeks except for the Perfume H. This perfume showed a discoloration at 6 weeks 40° C. only when tested with a starting formula that contained surfactant. Each perfume was tested in formulas without surfactant. (Standard formula was: 2% Dehyquart Guar HP, 3% glycerin, 0.9% preservative, 0.8% pH buffer, 0.6% perfume, 0.03% aloe.)

TABLE 16
HP Chassis no Surfactant

| Perfume | 5° C. | | | 25° C. | | | 40° C. | | |
|---|---|---|---|---|---|---|---|---|---|
| (0.6%) | Initial | 2 weeks | 6 weeks | Initial | 2 weeks | 6 weeks | Initial | 2 weeks | 6 weeks |
| Perfume A | Stable | Stable | Stable | Stable | Stable | Stable | Stable | Stable | Stable |
| Perfume B | Stable | Stable | Stable | Stable | Stable | Stable | Stable | Stable | Stable |
| Perfume C | Stable | Stable | Stable | Stable | Stable | Stable | Stable | Stable | Stable |
| Perfume D | Stable | Stable | Stable | Stable | Stable | Stable | Stable | Stable | Stable |
| Perfume E | Stable | Stable | Stable | Stable | Stable | Stable | Stable | Stable | Stable |
| Perfume F | Stable | Stable | Stable | Stable | Stable | Stable | Stable | Stable | Stable |
| Perfume G | Stable | Stable | Stable | Stable | Stable | Stable | Stable | Stable | Stable |
| Perfume H | Stable | Stable | Stable | Stable | Stable | Stable | Stable | Stable | Stable |

The results show that the formulations are able to solubilize skin actives and perfumes without surfactant and that these formulations remain soluble even after accelerated aging testing, after regular room temperature storage, and after cold storage (to replicate some shipping conditions). Stability was determined by visual checks for appearance and separation. Every 10 degree increase over RT is similar to a doubling in time, so 6 weeks at 45° C. considered an accelerated test that represents stability at Room Temp for 24 weeks (6 months).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to anything disclosed or claimed herein or that it alone, or in any combination with any other reference or references, teaches, suggests or discloses anything disclosed or claimed herein. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A method of removing sebum on hair, comprising:
wetting the hair with water;
applying to the wetted hair a shampoo composition comprising:
about 1% to about 10% cationic polymer, wherein the cationic polymer has a weight average molecular weight of about 400,000 to about 5,000,000, a charge density of about 0.4 meq/g to about 4 meq/g, and a surface tension of about 45 mN/m to about 70 mN/m;
wherein the composition has a viscosity of about 500 cps to about 30,000 cps; wherein the composition is substantially surfactant free; wherein the composition removes at least about 45% artificial sebum as measured by the Syringe Filter Polymer Cleaning procedure; and
rinsing the shampoo composition from the hair.

2. The method of claim 1, wherein the shampoo composition comprises about 1% to about 5% cationic polymer.

3. The method of claim 1, wherein the cationic polymer has a weight average molecular weight of about 1,000,000 to about 2,000,000.

4. The method of claim 1, wherein the shampoo composition has a surface tension of greater than about 60 mN/m.

5. The method of claim 1, wherein the shampoo composition has a surface tension of greater than about 70 mN/m.

6. The method of claim 1, wherein the shampoo composition removes at least about 50% or more artificial sebum.

7. The method of claim 1, wherein the shampoo composition removes at least about 60% or more artificial sebum.

8. The method of claim 1, further comprising one or more additional ingredients selected from viscosity modifying agents, perfume, scalp actives, opacifiers, sensates, feel actives, botanicals, vitamins, preservatives, humectants, sebum modifying actives, cosolvents, eutectics, or microcapsules and combinations thereof.

9. The method of claim 8, wherein the shampoo composition comprises a cosolvent.

10. The method of claim 1, wherein the shampoo composition is a foam.

11. The method of claim 1, wherein the shampoo composition is a solid.

12. The method of claim 1, wherein the cationic polymer is at least one of a homopolymer, a copolymer, a terpolymer, a branched polymer, a grafted polymer, or a cyclic polymer.

13. The method of claim 1, wherein at concentration 4000 ppm the shampoo composition has a level of TRPA1 Receptor activation that is <100 AUC, as determined by TRPA1 Cell Culture method.

14. The method of claim 1, wherein at concentration 4000 ppm the shampoo composition has a level of TRPV1 Receptor activation that is <100 AUC, as determined by TRPV1 Cell Culture method.

15. The method of claim 1, wherein at concentration 4000 ppm the shampoo composition has a level of TRPV3 Receptor activation that is <100 AUC, as determined by TRPV3 Cell Culture method.

16. The method of claim 1, wherein at concentration 4000 ppm the shampoo composition has a level of TRPm8 Receptor activation that is <100 AUC, as determined by TRPM8 Cell Culture method.
